(19) United States
(12) Patent Application Publication
(10) Pub. No.: US 2010/0144708 A1
(43) Pub. Date: Jun. 10, 2010

(54) HETEROCYCLIC COMPOUNDS CONTAINING NITROGEN ATOMS OR PHARMACEUTICALLY ACCEPTABLE SALTS THEREOF, PROCESS FOR THE PREPARATION THEREOF AND PHARMACEUTICAL COMPOSITION COMPRISING THE SAME FOR TREATMENT OF CANCER

(75) Inventors: Gyoon Hee Han, Gyeonggi-do (KR); Eun Hyun Choi, Seoul (KR); Jee Sun Yang, Seoul (KR); Sung Hee Hong, Gyeonggi-do (KR); Myung Sook Kim, Gangwon-do (KR); Mi Sun Won, Daejeon (JP); Kyung Sook Chung, Daejeon (KR); Dong Myung Kim, Daejeon (KR); Hyang Sook Yoo, Daejeon (KR); Hwan Mook Kim, Daejeon (KR); Song Kyu Park, Daejeon (KR); Ki Ho Lee, Chungbuk (KR); Chang Woo Lee, Chungbuk (KR); Youn Woong Choi, Gyeonggi-do (KR); Yong Mi Jeong, Seoul (KR); Byung Gu Min, Seoul (KR)

Correspondence Address: KNOBBE MARTENS OLSON & BEAR LLP, 2040 MAIN STREET, FOURTEENTH FLOOR, IRVINE, CA, 92614, US

(73) Assignees: Korea Research Instititute of Bioscience and Biotechnology, Daejeon (KR); Seoul Pharma, Co., Ltd., Seoul (KR)

(21) Appl. No.: 12/376,889

(22) PCT Filed: Aug. 10, 2007
(86) PCT No.: PCT/KR2007/003861

§ 371 Date: Mar. 20, 2009

(30) Foreign Application Priority Data
Aug. 10, 2006 (KR) 10-2006-0075827

(51) Int. Cl. A61K 31/551; C07D 243/08; C07D 211/96; A61K 31/4965; A61P 35/04

(52) U.S. Cl. 514/218; 540/575; 546/242; 514/255.02

(57) ABSTRACT

The present invention relates to new heterocyclic compounds containing nitrogen atoms or pharmaceutically acceptable salts thereof, a process for the preparation thereof, and a pharmaceutical composition comprising the same for treatment of cancer. The compounds according to the present invention induce DNA damage due to reactive oxygen species to activate c-abl and p53, induce RhoB to generate apoptosis, and induce cell death by down-regulating Bcl2 involved in cell survival, which is generated by dysregulated signals via the mitochondria pathway, thereby inhibiting tumor cell growth and inducing apoptosis. Accordingly, the composition according to the present invention can be used to treat cancer.

### TECHNICAL FIELD

The present invention relates to new heterocyclic compounds containing nitrogen atoms or pharmaceutically acceptable salts thereof, a process for the preparation thereof, and a pharmaceutical composition comprising the same for treatment of cancer.

### BACKGROUND ART

Cancer is characterized by uncontrolled cell growth, and the abnormal cell growth leads to the formation of a cell mass called a tumor. The cell mass invades nearby tissue, and in severe cases, may spread to other parts of the body. Cancer is academically called neoplasia.

Surgery, radiation therapy, and chemotherapy are provided for the treatment of cancer, but in many cases they cannot eradicate cancer. Further, cancer forces patients to suffer from pain, and ultimately leads to death, thereby being called an obstinate chronic disease. There are over 20 million patients suffering from cancer worldwide, of which 6 million or more die from cancer annually. Further, it is estimated that the number of cancer deaths could reach 11 million by 2020. Accordingly, cancer is a critical disease, of which treatment methods are needed to be urgently developed. Cancer represents 20% or more of the total causes of deaths in advanced countries and Korea, but the ratio is different for each country. Even though every effort has been made, the exact causes or mechanisms of cancer development have not yet been identified. There are many cancer-causing factors, and the factors can be classified into internal and external factors. The transformation mechanism of normal cells into cancer cells has not been identified, but the external factors including environmental factors have been known to account for at least 80-90% of the cancer-causing factors. The internal factors include immune conditions and inherited mutations, and the external factors include chemicals, radiation, and viruses. There are two types of genes involved in cancer development, oncogenes and tumor suppressor genes. When the above mentioned internal or external factors break the balance between the activities of oncogenes and tumor suppressor genes, cancer can be developed.

Cancer is largely classified into hematologic malignancies and solid tumors, and can develop in almost all parts of the body such as lung cancer, gastric cancer, breast cancer, oral cancer, liver cancer, uterine cancer, esophageal cancer, and skin cancer. Chemotherapy, which is one of the methods for treating such malignant tumors, excluding surgery and radiation therapy, is generally called an anti-cancer agent, and most of the anti-cancer agents are substances that mainly inhibit nucleic acid synthesis to exhibit anti-cancer activity.

Chemotherapy is largely divided into antimetabolites, alkylating agents, antimitotic drugs, hormones or the like. The antimetabolites inhibit the metabolism needed for the proliferation of cancer cells, and examples thereof include folic acid derivatives such as methotrexate, purine derivatives such as 6-mercaptopurine and 6-thioguanine, and pyrimidine derivatives such as 5-fluorouracil and cytarabine. The alkylating agents exhibit anti-cancer effects by introducing alkyl groups into guanine bases of the DNA to modify a DNA structure and cleave a DNA chain, and examples thereof include nitrogen mustard compounds such as chlorambucil and cyclophosphamide, ethyleneimine compounds such as thiotepa, alkylsulfonate compounds such as busulfan, nitrosourea compounds such as carmustine, and triazine compounds such as dacarbazine. The antimitotic drugs are cell cycle-specific drugs and block mitosis to inhibit cell division, and examples thereof include anti-cancer agents such as actinomycin D, doxorubicin, bleomycin, and mitomycin; plant alkaloids such as vincristine, vinblastine; and an antimitotic agent, taxane ring-containing toxoid. In addition, other anti-cancer agents include hormones such as adrenal cortical hormone and progesterone, and platinum-containing compounds such as cisplatin.

The biggest problem in chemotherapy is drug-resistance, which is the main reason the treatment eventually fails, despite the initial success of therapy with anti-cancer agents. A study to identify the reason why cancer becomes drug-resistance as well as development of anti-cancer agents with new mechanisms of action are continuously needed, in order to treat cancer that is resistant to the known agents. Anti-cancer agents currently under development include drug-resistance blocking agents, angiogenesis inhibitors, tumor metastasis inhibitors, and gene expression targeting drugs.

### DISCLOSURE OF INVENTION

### Technical Problem

Accordingly, the present inventors have made extensive studies on an anti-cancer agent with a new mechanism of action, which inhibits tumor proliferation and induces apoptosis. As a result, they have synthesized new heterocyclic compounds containing nitrogen atoms, and found that the compounds induce DNA damage due to reactive oxygen species to activate c-abl and p53, induce RhoB to generate apoptosis, and induce cell death by down-regulating Bcl2 involved in cell survival, which is generated by dysregulated signals via the mitochondria pathway, thereby completing the present invention.

### Technical Solution

The present invention provides new heterocyclic compounds containing nitrogen atoms or pharmaceutically acceptable salts thereof, process for the preparation thereof and a pharmaceutical composition comprising the same for treatment of cancer.

### BRIEF DESCRIPTION OF THE DRAWINGS

FIG. 1 is a drawing showing the body weight change of nude mice xenografted with a human prostate cancer cell line (PC-3), which had been intraperitoneally administered with the compound according to the present invention (Example 55).

FIG. 2 is a drawing showing the change in tumor size of nude mice xenografted with the human prostate cancer cell line (PC-3), which had been intraperitoneally administered with the compound according to the present invention (Example 55).

FIG. 3 is a drawing showing the tumor weights of nude mice xenografted with the human prostate cancer cell line (PC-3) on the final day (day 21), which had been intraperitoneally administered with the compound according to the present invention (Example 55).

FIG. 4 is a drawing showing the result of western blot analysis after treating the human prostate cancer cell line (PC-3) with the compound according to the present invention (Example 55).

FIG. 5 is a drawing showing the degree of apoptosis, after treating the human prostate cancer cell line (PC-3) with NAC (N-acetylcysteine), and then with the compound according to the present invention (Example 55).

### BEST MODE FOR CARRYING OUT THE INVENTION

The present invention provides heterocyclic compounds containing nitrogen atoms represented by Formula 1 or pharmaceutically acceptable salts thereof.

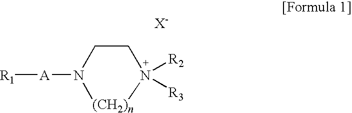

For the Formula 1,

R₁ is straight or branched chain C₁˜C₃₀ alkyl, or C₂˜C₃₀ alkenyl,

R₂ is straight or branched chain C₁˜C₆ alkyl,

R₃ is straight or branched chain C₁˜C₆ alkyl; C₂˜C₃₀ alkenyl; allyl; or benzyl substituted or unsubstituted with one group selected from the group consisting of straight or branched chain C₁˜C₆ alkyl, C₁˜C₆ alkoxy, OCF₃, nitro, and halogen atom,

A is C(═O) or S(═O)₂,

X is a halogen atom, and

n is an integer of 2 and 3.

Specific examples of the preferred compound among heterocyclic compounds containing nitrogen atoms of Formula 1 of the present invention are as follows:
1) 4-docosanoyl-1,1-dimethylpiperazin-1-ium iodide,
2) 1,1-dimethyl-4-octanoyl-piperazin-1-ium iodide,
3) 1-benzyl-1-methyl-4-octanoyl-piperazin-1-ium bromide,
4) 1-allyl-1-methyl-4-octanoyl-piperazin-1-ium bromide,
5) 1-(4-methoxy-benzyl)-1-methyl-4-octanoyl-piperazin-1-ium chloride,
6) 1-ethyl-1-methyl-4-octanoyl-piperazin-1-ium iodide,
7) 1-benzyl-1-ethyl-4-octanoyl-piperazin-1-ium bromide,
8) 1-benzyl-4-decanoyl-1-methyl-piperazin-1-ium bromide,
9) 1-allyl-4-decanoyl-1-methyl-piperazin-1-ium bromide,
10) 1-benzyl-4-decanoyl-1-ethyl-piperazin-1-ium bromide,
11) 1-allyl-4-decanoyl-1-ethylpiperazin-1-ium bromide,
12) 4-decanoyl-1-ethyl-1-methylpiperazin-1-ium iodide,
13) 1-allyl-1-methyl-4-tetradecanoyl-piperazin-1-ium bromide,
14) 1-ethyl-1-methyl-4-tetradecanoyl-piperazin-1-ium iodide,
15) 1-benzyl-1-ethyl-4-tetradecanoyl-piperazin-1-ium bromide,
16) 1-allyl-1-ethyl-4-tetradecanoyl-piperazin-1-ium bromide,
17) 1-allyl-4-hexadecanoyl-1-methyl-piperazin-1-ium bromide,
18) 4-hexadecanoyl-1-(4-methoxy-benzyl)-1-methyl-piperazin-1-ium chloride,
19) 4-hexadecanoyl-1-methyl-1-pent-4-enyl-piperazin-1-ium bromide,
20) 1-but-3-enyl-4-hexadecanoyl-1-methyl-piperazin-1-ium bromide,
21) 1-benzyl-1-ethyl-4-hexadecanoyl-piperazin-1-ium bromide,
22) 1-allyl-1-ethyl-4-hexadecanoyl-piperazin-1-ium bromide,
23) 1-ethyl-4-hexadecanoyl-1-methyl-piperazin-1-ium iodide,
24) 1-ethyl-4-hexadecanoyl-1-pent-4-enyl-piperazin-1-ium bromide,
25) 1-benzyl-1-methyl-4-octadecanoyl-piperazin-1-ium bromide,
26) 1-allyl-1-methyl-4-octadecanoyl-piperazin-1-ium bromide,
27) 1-ethyl-1-methyl-4-octadecanoyl-piperazin-1-ium iodide,
28) 1-benzyl-1-ethyl-4-octadecanoyl-piperazin-1-ium bromide,
29) 1-allyl-1-ethyl-4-octadecanoyl-piperazin-1-ium bromide,
30) 1-ethyl-4-icosanoyl-1-methylpiperazin-1-ium iodide,
31) 4-icosanoyl-1,1-dimethylpiperazin-1-ium iodide,
32) 1-benzyl-4-icosanoyl-1-methylpiperazin-1-ium bromide,
33) 1-allyl-4-icosanoyl-1-methylpiperazin-1-ium bromide,
34) 4-docosanoyl-1-ethyl-1-methylpiperazin-1-ium iodide,
35) 1-benzyl-4-docosanoyl-1-methylpiperazin-1-ium bromide,
36) 1-allyl-4-docosanoyl-1-methylpiperazin-1-ium bromide,
37) 1-benzyl-4-docosanoyl-1-ethylpiperazin-1-ium bromide,
38) 1-allyl-4-docosanoyl-1-ethylpiperazin-1-ium bromide,
39) 1,1-dimethyl-4-tetracosanoylpiperazin-1-ium iodide,
40) 1-benzyl-1-methyl-4-tetracosanoylpiperazin-1-ium bromide,
41) 1-allyl-1-methyl-4-tetracosanoylpiperazin-1-ium bromide,
42) 1-ethyl-1-methyl-4-tetracosanoylpiperazin-1-ium iodide,
43) 1-allyl-1-methyl-4-undec-10-enoyl-piperazin-1-ium bromide,
44) 1-benzyl-1-methyl-4-undec-10-enoyl-piperazin-1-ium bromide,
45) 1,1-dimethyl-4-undec-10-enoyl-piperazin-1-ium iodide,
46) 1-benzyl-1-methyl-4-palmitoylpiperazin-1-ium bromide,
47) 1-ethyl-1-(3-nitrobenzyl)-4-palmitoylpiperazin-1-ium bromide,
48) 1-ethyl-1-(4-fluorobenzyl)-4-stearoylpiperazin-1-ium bromide,
49) 1-ethyl-1-(3-nitrobenzyl)-4-stearoylpiperazin-1-ium bromide,
50) 1-(4-bromobenzyl)-1-ethyl-4-stearoylpiperazin-1-ium bromide,
51) 1-ethyl-1-(3-fluorobenzyl)-4-tetradecanoylpiperazin-1-ium bromide,
52) 1-ethyl-1-(3-methylbenzyl)-4-tetradecanoylpiperazin-1-ium bromide,
53) 4-dodecanoyl-1-ethyl-1-(3-nitrobenzyl)piperazin-1-ium bromide,
54) 4-dodecanoyl-1-ethyl-1-(3-fluorobenzyppiperazin-1-ium bromide,
55) 4-dodecanoyl-1-ethyl-1-(3-methylbenzyl)piperazin-1-ium bromide,
56) 1-ethyl-1-(3-nitrobenzyl)-4-tetradecanoylpiperazin-1-him bromide,
57) 1-ethyl-4-octadecyl-1-(3-(trifluoromethoxy)benzyl)piperazin-1-ium bromide,
58) 1-ethyl-1-(3-methylbenzyl)-4-stearoylpiperazin-1-ium bromide,
59) 1-ethyl-1-methyl-4-(octadecane-1-sulfonyl)piperazin-1-ium iodide,
60) 1-allyl-1-ethyl-4-(octane-1-sulfonyl)piperazin-1-ium bromide,
61) 1-allyl-1-methyl-4-(octane-1-sulfonyl)piperazin-1-ium bromide,
62) 1-benzyl-1-methyl-4-(octane-1-sulfonyl)piperazin-1-ium bromide,
63) 1-(4-fluorobenzyl)-1-methyl-4-(octane-1-sulfonyppiperazin-1-ium bromide,
64) 1-benzyl-1-ethyl-4-(undecane-1-sulfonyl)piperazin-1-ium bromide,
65) 1-benzyl-1-methyl-4-(undecane-1-sulfonyl)piperazin-1-ium bromide,
66) 1-(4-fluorobenzyl)-1-methyl-4-(undecane-1-sulfonyl)piperazin-1-ium bromide,
67) 1-allyl-1-ethyl-4-(tetradecane-1-sulfonyl)piperazin-1-ium bromide,
68) 1-ethyl-1-(4-fluorobenzyl)-4-(tetradecane-1-sulfonyl)piperazin-1-ium bromide,
69) 1-allyl-1-methyl-4-(tetradecane-1-sulfonyppiperazin-1-ium bromide,
70) 1-benzyl-1-methyl-4-(tetradecane-1-sulfonyl)piperazin-1-ium bromide,
71) 1-(4-fluorobenzyl)-1-methyl-4-(tetradecane-1-sulfonyl)piperazin-1-ium bromide,
72) 1-allyl-1-ethyl-4-(octadecane-1-sulfonyl)piperazin-1-ium bromide,
73) 1-benzyl-1-ethyl-4-(octadecane-1-sulfonyl)piperazin-1-ium bromide,
74) 1-allyl-1-methyl-4-(octadecane-1-sulfonyl)piperazin-1-ium bromide,
75) 4-(decane-1-sulfonyl)-1,1-dimethyl-piperazin-1-ium iodide,
76) 1-benzyl-4-(decane-1-sulfonyl)-1-methyl-piperazin-1-ium bromide,
77) 1-allyl-4-(decane-1-sulfonyl)-1-methyl-piperazin-1-ium bromide,
78) 4-(decane-1-sulfonyl)-1-ethyl-1-methyl-piperazin-1-ium iodide,
79) 1-benzyl-4-(decane-1-sulfonyl)-1-ethyl-piperazin-1-ium bromide,
80) 1-allyl-4-(decane-1-sulfonyl)-1-ethyl-piperazin-1-ium bromide,
81) 1-benzyl-4-(dodecane-1-sulfonyl)-1-methyl-piperazin-1-ium bromide,
82) 1-allyl-4-(dodecane-1-sulfonyl)-1-methyl-piperazin-1-ium bromide,
83) 4-(dodecane-1-sulfonyl)-1-ethyl-1-methyl-piperazin-1-ium iodide,
84) 1-benzyl-4-(dodecane-1-sulfonyl)-1-ethyl-piperazin-1-ium bromide,
85) 1-allyl-4-(dodecane-1-sulfonyl)-1-ethyl-piperazin-1-ium bromide,
86) 4-(decane-1-sulfonyl)-1-(4-fluorobenzyl)-1-methyl-piperazin-1-ium bromide,
87) 4-(decane-1-sulfon yl)-1-ethyl-1-(4-fluorobenzyl)-piperazin-1-ium bromide,
88) 4-(dodecane-1-sulfonyl)-1-(4-fluorobenzyl)-1-methyl-piperazin-1-ium bromide,
89) 1-ethyl-1-(3-nitrobenzyl)-4-(nonylsulfonyl)piperazin-1-ium bromide,
90) 4-(dodecane-1-sulfon yl)-1-methyl-1-(4-methylbenzyl)piperazin-1-ium bromide,
91) 4-(decane-1-sulfonyl)-1-ethyl-1-(3-nitrobenzyl)piperazin-1-ium bromide,
92) 1,1-diethyl-4-(undecane-1-sulfonyl)piperazin-1-ium iodide,
93) 1-ethyl-1-methyl-4-(undecane-1-sulfonyl)piperazin-1-ium iodide,
94) 1,1-diethyl-4-(tetradecane-1-sulfonyl)piperazin-1-ium iodide,
95) 1-ethyl-1-methyl-4-(tetradecane-1-sulfonyl)piperazin-1-ium iodide,
96) 1-(4-fluorobenzyl)-1-methyl-4-(octadecane-1-sulfonyl)piperazin-1-ium bromide,
97) 4-(dodecane-1-sulfonyl)-1-ethyl-1-(4-fluorobenzyl)piperazin-1-ium bromide,
98) 4-(dodecane-1-sulfonyl)-1-ethyl-1-(3-nitrobenzyl)piperazin-1-ium bromide,
99) 1-ethyl-1-methyl-4-(nonane-1-sulfonyl)piperazin-1-ium iodide,
100) 1-benzyl-1-ethyl-4-(nonane-1-sulfonyl)piperazin-1-ium bromide,
101) 1-allyl-1-ethyl-4-(nonane-1-sulfonyl)piperazin-1-ium bromide,
102) 1,1-dimethyl-4-(nonane-1-sulfonyl)piperazin-1-ium iodide,
103) 1-benzyl-1-methyl-4-(nonane-1-sulfonyl)piperazin-1-ium bromide,
104) 1-(4-fluorobenzyl)-1-methyl-4-(nonane-1-sulfonyl)piperazin-1-ium bromide,
105) 4-(decane-1-sulfonyl)-1-methyl-1-(3-nitrobenzyl)piperazin-1-ium bromide,
106) 4-(decane-1-sulfonyl)-1-methyl-1-(3-methylbenzyl)piperazin-1-ium bromide,
107) 4-(decane-1-sulfonyl)-1-methyl-1-(4-methylbenzyl)piperazin-1-ium bromide,
108) 4-(dodecane-1-sulfonyl)-1-methyl-1-(3-methylbenzyl)piperazin-1-ium bromide,
109) 4-(decane-1-sulfonyl)-1-ethyl-1-(4-methylbenzyl)piperazin-1-ium bromide,
110) 4-(decane-1-sulfonyl)-1-ethyl-1-(3-methylbenzyl)piperazin-1-ium bromide,
111) 1-benzyl-1-ethyl-4-(octane-1-sulfonyl)piperazin-1-ium bromide,
112) 1-allyl-1-methyl-4-(undecane-1-sulfonyl)piperazin-1-ium bromide,
113) 1-benzyl-1-ethyl-4-(tetradecane-1-sulfonyl)piperazin-1-ium bromide,
114) 1-(4-t-butylbenzyl)-1-ethyl-4-(octadecane-1-sulfonyl)piperazin-1-ium bromide,
115) 1-benzyl-1-methyl-4-(octadecane-1-sulfonyl)piperazin-1-ium bromide,
116) 1-ethyl-1-methyl-4-palmitoyl-1,4-diazepan-1-ium iodide,
117) 4-dodecanoyl-1,1-dimethyl-1,4-diazepan-1-ium iodide,
118) 1-allyl-4-dodecanoyl-1-methyl-1,4-diazepan-1-ium bromide,
119) 4-dodecanoyl-1-(4-methoxybenzyl)-1-methyl-1,4-diazepan-1-ium chloride,
120) 1-allyl-1-methyl-4-palmitoyl-1,4-diazepan-1-ium bromide,
121) 1-(4-fluorobenzyl)-1-methyl-4-palmitoyl-1,4-diazepan-1-him bromide,
122) 4-hexadecanoyl-1,1-dimethyl-1,4-diazepan-1-ium iodide,
123) 1,1-dimethyl-4-tetradecanoyl-1,4-diazepan-1-ium iodide,
124) 1-benzyl-1-methyl-4-tetradecanoyl-1,4-diazepan-1-ium bromide,
125) 1-benzyl-1-methyl-4-palmitoyl-1,4-diazepan-1-ium bromide,
126) 4-hexadecyl-1-methyl-1-(3-(trifluoromethoxy)benzyl)-1,4-diazepan-1-ium bromide,
127) 1-allyl-1-methyl-4-tetradecanoyl-1,4-diazepan-1-ium bromide,
128) 1-methyl-1-(3-nitrobenzyl)-4-tetradecanoyl-1,4-diazepan-1-ium bromide,
129) 1,1-dimethyl-4-stearoyl-1,4-diazepan-1-ium iodide,
130) 1-(4-fluorobenzyl)-1-methyl-4-stearoyl-1,4-diazepan-1-ium bromide,
131) 1-allyl-1-methyl-4-octadecyl-1,4-diazepan-1-ium bromide,
132) 1-methyl-1-(3-methylbenzyl)-4-octadecyl-1,4-diazepan-1-ium bromide,
133) 1-(4-fluorobenzyl)-1-methyl-4-tetradecanoyl-1,4-diazepan-1-ium bromide,
134) 1-(4-fluorobenzyl)-1-methyl-4-octadecyl-1,4-diazepan-1-ium bromide, and
135) 1-methyl-1-(3-nitrobenzyl)-4-octadecyl-1,4-diazepan-1-ium bromide.

The compounds of the present invention may be prepared in the forms of pharmaceutically acceptable salts and solvates according to the known method in the related art.

As the pharmaceutically acceptable salts, acid addition salts produced with free acids are preferred. The acid addition salts are prepared by the known method, for example, a method including the steps of dissolving a compound in an excessive amount of acid aqueous solution, and precipitating the salt using a water-miscible organic solvent such as methanol, ethanol, acetone or acetonitrile. Acid or alcohol (e.g. glycol monomethyl ether) in the same molar amount of compound and water is heated, and the mixture is dried by evaporation or the precipitated salt can be suction-filtered.

At this time, as the free acids, organic acids and inorganic acids may be used. Examples of the inorganic acids include hydrochloric acid, phosphoric acid, sulfuric acid, nitric acid, and tartaric acid, and examples of the organic acids include methanesulfonic acid, p-toluenesulfonic acid, acetic acid, trifluoroacetic acid, citric acid, maleic acid, succinic acid, oxalic acid, benzoic acid, tartaric acid, fumaric acid, mandelic acid, propionic acid, lactic acid, glycollic acid, gluconic acid, galacturonic acid, glutamic acid, glutaric acid, glucuronic acid, aspartic acid, ascorbic acid, carboxylic acid, vanillic acid, and hydroiodic acid, but are not limited thereto.

Further, pharmaceutically acceptable metal salts can be prepared using a base. An alkali metal salt and alkaline earth metal salt can be obtained by, for example, the method including the steps of dissolving a compound in an excessive amount of alkali metal hydroxide or alkaline earth metal hydroxide solution, filtering the undissolved salt, and then evaporating and drying the filtrate. In respects to metal salts, sodium, potassium, or calcium salt is pharmaceutically preferable, and the corresponding silver salt is obtained by reacting alkali metal salt or alkaline earth metal salt with a suitable silver salt (e.g. silver nitrate).

A pharmaceutically acceptable salt of the compound represented by Formula 1 includes salts of acidic or basic groups, which can be present in the compound of Formula 1, as long as particular mention is not made. For example, the pharmaceutically acceptable salt includes sodium salt, calcium salt, and potassium salt of hydroxy group, and other pharmaceutically acceptable salt of amino group includes hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate). Further, the salts can be prepared by a preparation method or preparation process thereof known in the related art.

Further, the present invention provides a process for the preparation of the heterocyclic compound containing nitrogen atoms of Formula 1, which is represented by Reaction Schemes 1 to 3.

In the compound of Formula 1, if A is C(═O) and n is 2, the method can be performed as shown in Reaction Scheme 1. The method includes the steps of:

1) reacting the organic acid compound of Formula 2 with thionyl chloride in an organic solvent, and then reacting with an alkylpiperazine derivative to prepare the compound of Formula 3; and

2) reacting the compound of Formula 3 prepared in the step 1) with a halide compound to prepare the compound of Formula 1-1.

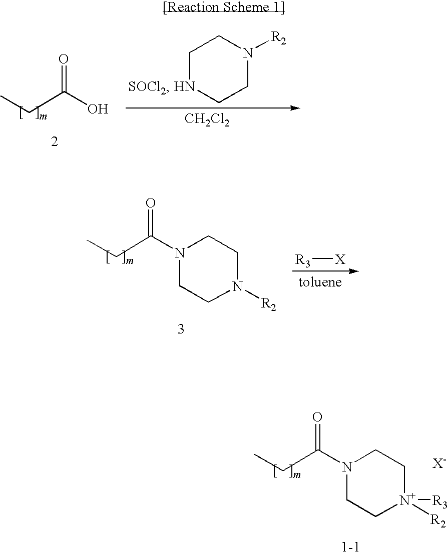

For Reaction Scheme 1, R₂, R₃ and X are as defined in Formula 1, and m is an integer of 1 to 30.

Further, in the compounds of Formula 1, if A is S(═O)₂ and n is 2, the method can be performed as shown in Reaction Scheme 2. The method includes the steps of:

1) reacting the sulfonic acid compound of Formula 4 with oxalyl chloride in an organic solvent, and then reacting with an alkylpiperazine derivative to prepare the compound of Formula 5; and

2) reacting the compound of Formula 5 prepared in the step 1) with a halide compound to prepare the compound of Formula 1-2.

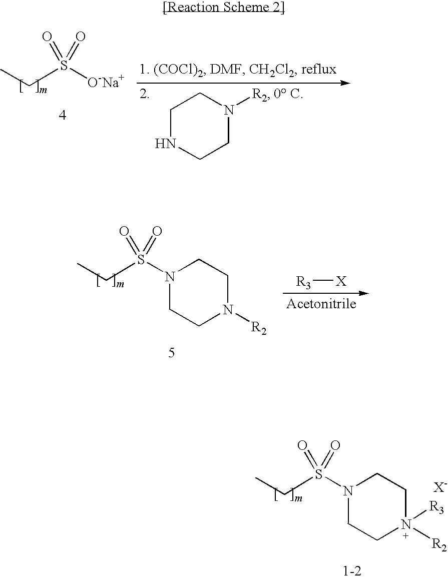

For Reaction Scheme 2, R₂, R₃ and X are as defined in Formula 1, and m is an integer of 1 to 30.

Further, in the compounds of Formula 1, if A is C(═O) and n is 3, the method can be performed as shown in Reaction Scheme 3. The method includes the steps of:

1) reacting the organic acid compound of Formula 2 with an alkyldiazepan derivative in an organic solvent to prepare the compound of Formula 6; and

2) reacting the compound of Formula 6 prepared in the step 1) with a halide compound to prepare the compound of Formula 1-3.

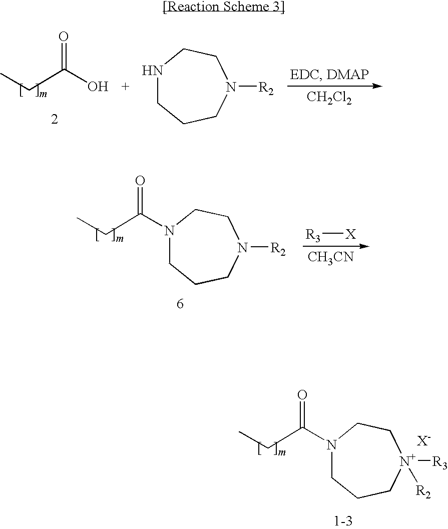

For Reaction Scheme 3, R₂, R₃ and X are as defined in Formula 1, and m is an integer of 1 to 30.

The method for the preparation of the compounds of Formula 1 of the present invention will be specifically described as follows.

Reaction Schemes 1 to 3 represent the two-step process of the preparation of the compound of Formula 1, in which the compound of Formula 1 is prepared using a commercially available organic acid compound of Formula 2 and sulfonic acid compound of Formula 4 as starting materials.

In the step 1) of Reaction Schemes 1 to 3, the organic acid compound of Formula 2 or sulfonic acid compound of Formula 4 is reacted with thionyl chloride or oxalyl chloride in an organic solvent in a temperature range of room temperature to 60° C. to prepare organic acid chloride or sulfonic acid chloride as an intermediate, and then reacted with an alkylpiperazine derivative at 0° C. to prepare the compound of Formula 3 or the compound of Formula 5. Alternatively, the organic acid compound of Formula 2 is reacted with an alkyldiazepan derivative in an organic solvent to prepare the compound of Formula 6.

At this time, methylene chloride or the like can be used as the organic solvent. 2 to 4 equivalent weights of thionyl chloride or oxalyl chloride can be used, based on the organic acid compound of Formula 2 or sulfonic acid compound of Formula 4 as starting materials, and 4 equivalent weights of alkylpiperazine can be used, based on the organic acid compound of Formula 2 or sulfonic acid compound of Formula 4.

In the step 2) of Reaction Schemes 1 to 3, the compound of Formula 3 or the compound of Formula 5 or the compound of Formula 6 prepared in the step 1) is reacted with a halide compound in an organic solvent to prepare the compounds of Formulae 1-1 to 1-3. At this time, toluene, benzene, acetonitrile or the like can be used as the organic solvent. Further, it is preferable that the halide compound is methyliodide, benzylbromide, allylbromide or the like, and 2 to 3 equivalent weights thereof can be used, based on the compound of Formula 3 or the compound of Formula 5 or the compound of Formula 6, and the reaction can be performed in a temperature range of room temperature to 100° C.

Further, the present invention provides a pharmaceutical composition for treatment of cancer that includes the heterocyclic compounds containing nitrogen atoms of Formula 1 or pharmaceutically acceptable salts thereof as an active ingredient.

Examples of cancer include lung cancer, non-small cell lung cancer, colon cancer, bone cancer, pancreatic cancer, skin cancer, head or neck cancer, cutaneous or ocular melanoma, uterine cancer, ovarian cancer, rectal cancer, gastric cancer, anal cancer, breast cancer, fallopian tube carcinoma, endometrial carcinoma, cervical carcinoma, vaginal carcinoma, vulvar carcinoma, Hodgkin's disease, esophageal cancer, small intestine cancer, endocrine gland cancer, thyroid cancer, parathyroid cancer, adrenal cancer, soft-tissue sarcoma, uterine cancer, penis cancer, prostate cancer, chronic or acute leukemia, lymphocyte lymphoma, bladder cancer, kidney or ureter cancer, renal cell carcinoma, renal pelvic carcinoma, central nervous system tumor, primary central nervous system lymphoma, spinal tumor, brain stem glioma, and pituitary adenoma.

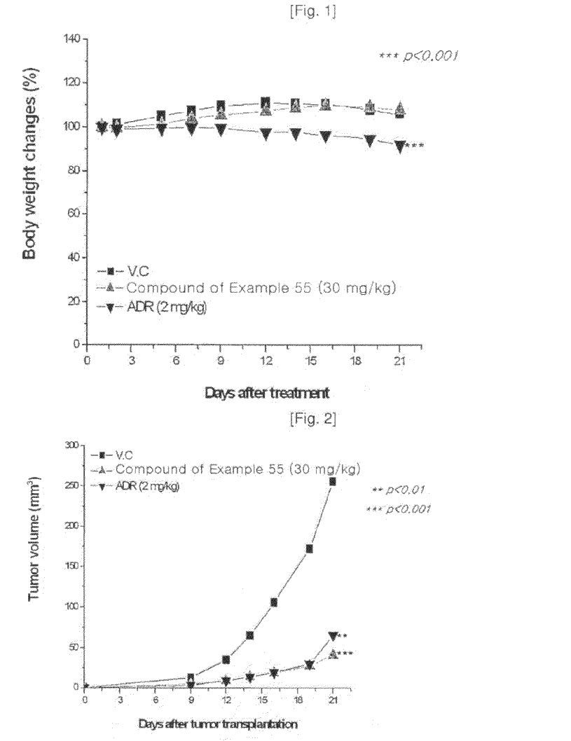

Specific symptoms and weight loss were not observed in nude mice xenografted with human prostate cancer cell line, PC-3 cells, which had been intraperitoneally administered with the compound of the present invention (30 mg/kg), for the experimental period. Further, a statistically significant effect of inhibiting tumor growth was observed (84.0%, p<0.001), and a statistically significant effect was observed in the reduction of tumor weight (79.5%, p<0.001) on the final day of the experiment (day 21) (FIGS. 1 to 3).

Further, a human prostate cancer cell line PC-3 was treated with the compound according to the present invention, and then the amount of protein was measured by Western blotting. As a result, the amount of c-abl in response to DNA damage increased, and the amount of p53 and phosphorylated p53 sharply increased. Further, the amount of RhoB increased, which has been reported to be involved in apoptosis, and apoptosis was generated by the RhoB induction (FIG. 4A). Further, the amount of Bcl2 involved in the cell survival was down-regulated by dysregulated signals via the mitochondria pathway (FIG. 4B).

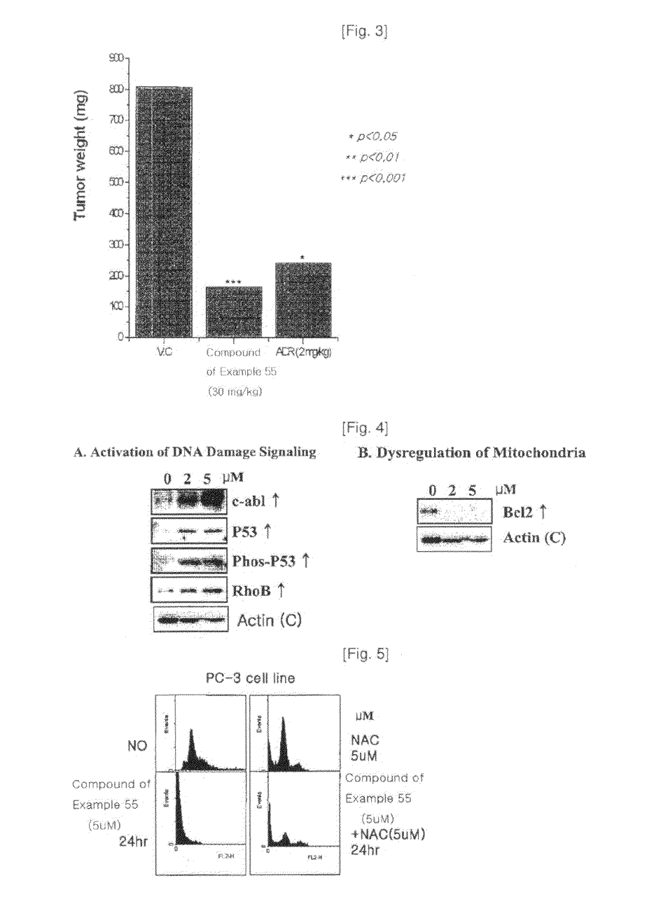

Further, when the human prostate cancer cell line PC-3 was treated with an antioxidant NAC (N-acetylcysteine) that functions to remove reactive oxygen species (ROS), the degree of apoptosis was greatly decreased, as compared to the prostate cancer cell line PC-3 treated with only the compound according to the present invention (FIG. 5).

Therefore, the compounds according to the present invention induce DNA damage due to reactive oxygen species to activate c-abl and p53, induce RhoB to generate apoptosis, and induce cell death by down-regulating Bcl2 involved in the cell survival, which is generated by dysregulated signals via the mitochondria pathway, thereby inhibiting tumor cell growth and inducing apoptosis. Accordingly, the composition according to the present invention can be used to treat cancer.

The pharmaceutical composition comprising the compounds of Formula 1 according to the present invention can further contain a suitable carrier, excipient, or diluent according to the conventional method. Examples of the carrier, excipient, and diluent contained in the composition of the present invention include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oils.

The composition according to the present invention may be formulated into an oral preparation such as a powder, a granule, a tablet, a capsule, a suspension, an emulsion, a syrup, and an aerosol, an external preparation, suppository, or a sterilized injectable solution according to a conventional method.

Specifically, such preparations are prepared using diluents or excipients ordinarily employed, such as filler, extender, binder, wetting agent, disintegrating agent, and surfactant. Examples of the solid preparation for oral administration include a tablet, a pill, a powder, a granule, and a capsule, and the solid preparation can be prepared by mixing the compound with at least one excipient such as starch, calcium carbonate, sucrose, lactose, and gelatin. Further, in addition to the excipients, lubricants such as magnesium stearate and talc can be used. Examples of a liquid preparation for oral administration include a suspension, a liquid for internal use, an emulsion, a syrup or the like, and various excipients such as wetting agent, sweetener, flavor, and preservative can be contained, in addition to general diluents such as water and liquid paraffin. Examples of the preparation for parenteral administration include an aseptic aqueous solution, a non-aqueous solvent, suspension, emulsion, a lyophilized agent, and suppository. As the non-aqueous solvent and suspension, propylene glycol, polyethylene glycol, plant oil such as olive oil, injectable ester such as ethyloleate or the like can be used. As a suppository base, witepsol, macrogol, tween 61, cacao butter, lauric butter, glycerogelatin or the like can be used.

The preferred dosage of the composition according to the present invention can vary depending on various factors, including the patient's condition and body weight, disease severity, drug formulation, administration route and time, and can be suitably selected by those skilled in the art. However, for better efficacy, the compound of the present invention can be administered at a daily dosage of 0.0001 to 100 mg/kg, preferably 0.001 to 100 mg/kg once or several times. The compound of Formula 1 in the composition of the present invention should be present in an amount of 0.0001 to 10% by weight, preferably 0.001 to 1% by weight based on the total weight of the composition.

The compound according to the present invention can be used in the form of pharmaceutically acceptable salt thereof, and singly or collectively, as well as in combination with other pharmaceutically active compounds.

The pharmaceutical composition of the present invention can be administered to mammals such as rats, mice, domestic animals, and human via various routes. Any administration route can be considered, and the composition can be administered, for example, by oral, rectal or intravenous injection, intramuscular injection, subcutaneous injection, and epidural or intracerebroventricular injection.

### MODE FOR THE INVENTION

Hereinafter, the present invention will be described in more detail with reference to Examples. However, these Examples are for the illustrative purpose only, and the invention is not intended to be limited by these Examples.

### Example 1

### Preparation of 4-docosanoyl-1,1-dimethylpiperazin-1-ium iodide according to Reaction Scheme 1

### 1. Preparation of 1-(4-methylpiperazine-1-yl)docosan-1-one

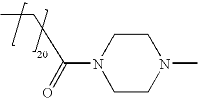

Thionyl chloride (0.43□, 5.86 mmol) was added to a 0.1 M methylene chloride solution of docosanoic acid (1 g, 2.93 mmol) under stirring, and heated for 4 hours. The produced mixture was cooled to room temperature, and then methylpiperazine (1.30□, 11.72 mmol) was added thereto at 0° C. under stirring for 2 hours. The produced mixture was diluted with chloroform, and then washed with 10% NaOH (added up to pH 13). The mixture was washed with a saturated brine solution, the organic layer was dried over magnesium sulfate, and distilled off under reduced pressure. The resulting primary compound was purified by a silica gel column chromatography (eluent: 5% methanol/chloroform) to obtain the target compound in 25% yield (307 mg). (The reaction using ethylpiperazine instead of methylpiperazine was performed as the above method.)

¹H-NMR (400 MHz, DMSO) δ 3.64-3.48 (m, 4H), 2.41-2.36 (m, 4H), 2.33-2.29 (m, 2H) 2.31 (s, 3H), 1.62 (br m, 2H), 1.25 (br s, 36H), 0.88 (t, 3H, J=6.8 Hz).

### 2. Preparation of 4-docosanoyl-1,1-dimethylpiperazin-1-ium iodide

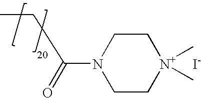

Iodomethane (0.02□, 0.3 mmol) was added to a 0.1 M toluene solution of the compound (80 mg, 0.19 mmol) obtained in the step 1 under stirring, and heated for 3 hours. The produced mixture was sufficiently cooled to 0° C., and then an ethyl acetate solution (6.0□) was added thereto under stirring for 2 hours. The produced mixture was filtered with eluding ethyl acetate solvent to obtain the target compound in 73.6% yield (81 mg).

¹H-NMR (300 MHz, DMSO) δ 3.76-3.37 (m, 8H), 3.12 (s, 6H), 2.32 (t, 2H, J=7.4 Hz), 1.43 (br m, 2H), 1.22 (br s, 36H), 0.84 (t, 3H, J=5.9 Hz).

### Examples 2 to 58

The compounds of Examples 2 to 58 were prepared in a similar manner to the preparation process that is described in Example 1.

The physical properties of the compounds are shown in Table 1.

TABLE 1

| | | MS data | |
|---|---|---|---|
| Exam- | | (M⁺H), | |
| ple | Chemical structure | (g/mol) | ¹H NMR spectrum data |
| 2 | 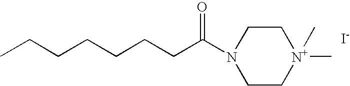 | 242(−I) | 3.81 (t, 2H, J = 4.6 Hz), 3.77 (t, 2H. J = 4.6 Hz), 3.43 (t, 2H, J = 4.8 Hz), 3.36 (t, 2H, 4.8 Hz), 3.18 (s, 6H), 2.35 (t, 2H, J = 7.4 Hz), 1.49 (br m, 2H) 1.27 (br m, 8H) 0.86 (t, 3H, J = 6.8 Hz) |
| 3 | 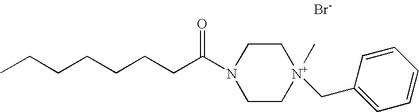 | 318(−Br) | 7.59-7.53 (m, 5H), 4.75 (s, 2H), 4.19 (d, 1H, J = 13.6 Hz), 4.03 (d, 1H, J = 14.4 Hz), 3.74 (t, 1H, J = 11.6 Hz), 3.54-3.36 (m, 5H), 3.06 (s, 3H), 2.51-2.33 (m, 2H), 1.49 (br m, 2H), 1.27 (br m, 8H), 0.87 (t, 3 H, J = 6.4 Hz) |
| 4 | 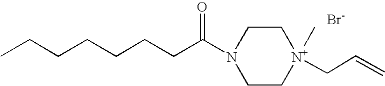 | 268(−Br) | 6.13-6.03 (m, 1H), 5.71-5.65 (m, 2H), 4.17 (d, 2H, J = 7.2 Hz), 3.96-3.36 (m, 8H), 3.11 (s, 3H), 2.35 (t, 2H, J = 7.4 Hz), 1.49 (br m, 2H), 1.27 (br m, 8H), 0.87 (t, 3H, J = 6.2 Hz) |
| 5 | 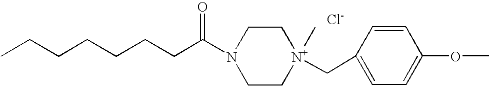 | 348(−Cl) | 7.51 (d, 2H, J = 8.8 Hz), 7.06 (d, 2H, J = 8.8 Hz), 4.70 (s, 2H), 3.81 (s, 3H), 3.75- 3.31 (m, 8H), 3.04 (s, 3H), 2.42-2.29 (m, 2H), 1.51-1.47 (m, 2H), 1.27-1.18 (m, 8H), 0.87 (t, 3H, J = 6.8 Hz) |
| 6 | 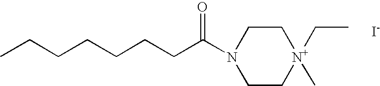 | 256(−I) | 3.63-3.36 (m, 8H), 2.45-2.36 (m, 5H), 2.28 (t, 2H, J = 7.6 Hz), 1.61-1.55 (m, 2H), 1.26-1.24 (m, 8H), 1.07 (t, 3H, J = 7.2 Hz), 0.86-0.76 (m, 3H) |
| 7 | 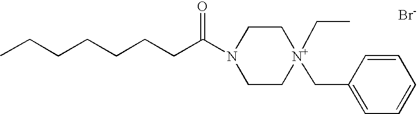 | 332(−Br) | 7.91-7.47 (m, 5H), 4.79-4.70 (m, 2H), 3.59-3.38 (m, 4H), 3.26-3.12 (m, 4H), 2.79-2.63 (m, 2H), 1.56-1.46 (m, 5H), 1.29-1.25 (m, 8H), 0.88-0.80 (m, 3H) |
| 8 | 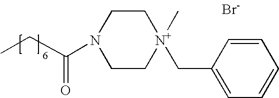 | 346(−Br) | 7.55-7.51 (m, 5H), 4.67 (s, 2H), 3.50-3.38 (m, 8H), 3.30 (s, 3H), 2.37-2.33 (m, 2H), 1.52-1.46 (m, 2H), 1.30-1.22 (m, 12H), 0.86 (t, 3H, J = 6.0 Hz) |
| 9 | 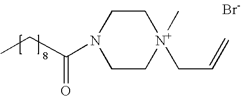 | 295(−Br) | 6.12-6.02 (m, 1H), 5.66 (t, 2H, J = 11.6 Hz), 4.15 (d, 2H, J = 4 Hz), 3.91 (t, 2H, J = 14.2 Hz). 3.80-3.74 (m, 1H), 3.66- 3.61 (m, 1H), 3.41 (t, 4H, J = 5.0 Hz), 3.10 (s, 3H), 2.34 (t, 2H, J = 7.4 Hz), 1.40-1.49 (m, 2H), 1.31-1.19 (m, 12H), 0.85 (t, 3H, J = 6.4 Hz) |
| 10 | 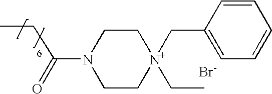 | 360(−Br) | 7.57-7.51 (m, 5H), 4.68 (s, 2H), 4.14-3.49 (m, 4H), 3.41-3.29 (m, 6H), 2.39-2.29 (m, 2H), 1.48 (br m, 2H), 1.38 (t, 3H, J = 7.2 Hz), 1.25 (br s. 12H), 0.86 (t, 3H, J = 6.4 Hz) |
| 11 | 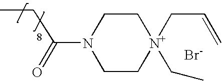 | 310(−Br) | 6.01-5.97 (m, 1H), 5.72-5.62 (m, 2H), 4.09 (d, 2H, J = 7.2 Hz), 3.81-3.78 (m, 4H), 3.45-3.37 (m, 4H), 2.34 (t, 2H, J = 7.6 Hz), 1.48 (br m, 2H), 1.24 (br s, 15H), 0.86 (t, 3H, J = 6.4 Hz) |
| 12 | 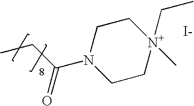 | 284(−I) | 3.94-3.28 (m, 10H), 3.05 (s, 3H), 2.32 (t, 2H, J = 7.4 Hz), 1.46 (br s, 2H), 1.23 (br s, 15H), 0.84 (t, 3H J = 6.0 Hz) |
| 13 | 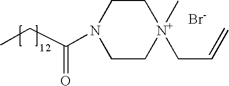 | 351(−Br) | 6.09-6.00 (m, 1H), 5.64 (d, 2H, J = 5.7 Hz), 4.07 (t, 2H, J = 8.3 Hz), 3.89 (t, 2H, J = 15.0 Hz), 3.76 (s, 3H), 3.64-3.36 (m, 2H), 3.13 (t, 2H, J = 30.6 Hz), 2.32 (t, 2H, J = 7.2 Hz), 1.50-1.40 (m, 2H), 1.30- 1.15 (m, 20H), 0.84 (t, 3H, J = 6.3 Hz) |
| 14 | 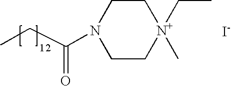 | 339(−I) | 3.81 (t, 4H, J = 15.0 Hz), 3.49-3.41 (m, 2H), 3.32-3.26 (m, 4H), 3.04 (s, 3H), 2.32 (t, 2H, J = 7.2 Hz), 1.50-1.40 (m, 2H), 1.30-1.18 (m, 13H), 0.83 (t, 3H, J = 6.0 Hz) |
| 15 | 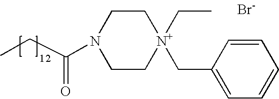 | 415(−Br) | 7.45-6.82 (m, 5H), 4.70 (d, 2H, J = 4.5 Hz), 4.15-4.02 (m, 2H), 3.97-3.85 (m, 2H), 3.62-3.41 (m, 4H), 3.12-3.03 (m, 2H), 2.71-2.61 (m, 2H), 2.30-2.20 (m, 2H), 1.47-1.17 (m, 20H), 0.81 (t, 3H, J = 6.5 Hz) |
| 16 | 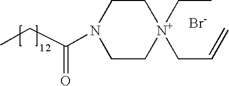 | 365(−Br) | 6.01-5.95 (m, 1H), 5.73-5.60 (m, 2H), 4.25 (d, 2H, J = 4.2 Hz), 4.06 (d, 2H, J = 3.6 Hz), 3.79-3.70 (m, 4H), 3.50-3.31 (m, 4H), 2.32 (t, 2H, J = 7.4 Hz), 2.46 (br m, 2H), 1.50-1.23 (m, 23H), 0.84 (t, 3H, J = 6.6 Hz) |
| 17 | 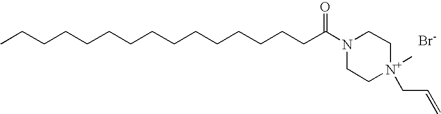 | 380(−Br) | 6.13-6.03 (m, 1H), 5.70-5.65 (m, 2H), 4.16 (d, 2H, J = 6.8 Hz), 3.96-3.63 (m, 4H), 3.42-3.36 (m, 4H), 3.11 (s, 3H), 2.35 (t, 2H, J = 7.2 Hz), 1.48 (br m. 2H), 1.24 (br m. 24H), 0.86 (t, 3H, J = 6.2 Hz) |
| 18 | 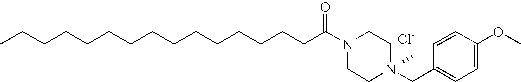 | 460(−Cl) | 7.50 (d, 2H, J = 8.8 Hz), 7.07 (d, 2H, J = 8.8 Hz), 4.68 (s, 2H), 3.81 (s, 3H), 3.76- 3.34 (m, 8H), 3.04 (s, 3H), 2.39-2.30 (m, 2H), 1.48-1.47 (m, 2H), 1.24 (br m, 24H), 0.86 (t, 3H), J = 6.8 Hz) |
| 19 | 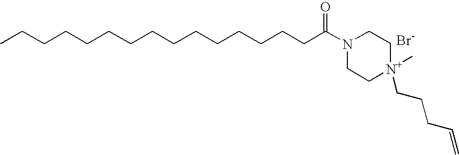 | 408(−Br) | 5.89-5.78 (m, 1H), 5.14-5.02 (m, 2H), 4.08-3.32 (m, 8H), 3.18 (s, 3H), 3.13- 3.11 (m, 2H), 2.34 (t, 2H, J = 7.4 Hz), 2.18-2.05 (m, 2H), 1.84-1.79 (m, 2H), 1.66-1.64 (m, 2H), 1.40-1.09 (m, 24H), 0.87-0.84 (m, 3H) |
| 20 | 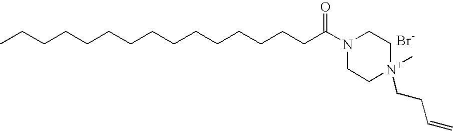 | 394(−Br) | 5.81-5.70 (m, 1H), 5.25-5.12 (m, 2H), 3.95-3.38 (m, 10H), 3.13 (s, 3H), 2.32 (t, 2H, J = 7.4 Hz), 1.46 (br m, 2H), 1.22 (br s, 26H), 0.83 (t, 3H, J = 6.6 Hz) |
| 21 | 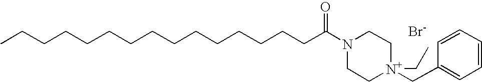 | 444(−Br) | 7.52-7.48 (m, 5H), 4.13-3.36 (m, 10H), 2.35-2.28 (m, 2H), 1.45-1.43 (m, 2H), 1.36 (t, 3H, J = 7.0 Hz), 1.22 (br s, 24H), 0.83 (t, 3H, J = 6.6 Hz) |
| 22 | 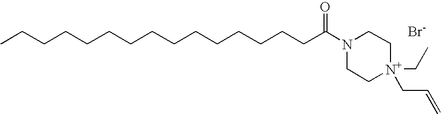 | 394(−Br) | 6.27-5.91 (m, 1H), 5.85-5.69 (m, 2H), 4.44-4.21 (m, 2H) 4.02-3.70 (m, 8H), 3.57 (br s, 2H), 2.40-2.35 (m, 2H), 1.59-1.56 (m, 2H), 1.49 (t, 3H, J = 6.9 Hz), 1.32- 1.16 (m, 24H), 0.87 (t, 3H, J = 6.5 Hz) |
| 23 | 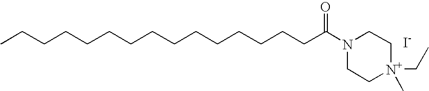 | 368(−I) | 3.89-3.35 (m, 10H), 3.05 (s, 3H), 2.32 (t, 2H, J = 7.4 Hz), 1.48-1.44 (m, 2H), 1.26- 1.22 (m, 27H), 0.83 (t, 3H, J = 6.7 Hz) |
| 24 | 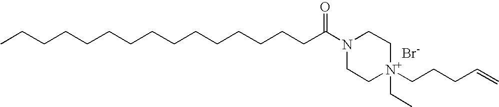 | 422(−Br) | 5.87-5.78 (m, 1H), 5.13-5.01 (m, 2H), 3.77-3.73 (m, 4H), 3.50-3.35 (m, 8H), 2.31 (t, 2H, J = 7.4 Hz), 2.08-2.03 (m, 2H), 1.72-1.67 (m, 2H), 1.46-1.44 (m, 2H), 1.22-1.15 (m, 27H), 0.83 (t, 3H, J = 6.6 Hz) |
| 25 | 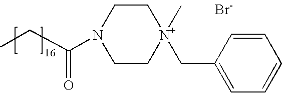 | | 7.56-7.51 (m, 5H), 4.71 (s, 2H), 4.20-3.38 (m, 8H), 3.02 (s, 3H), 2.37-2.32 (m, 2H), 1.23-1.15 (m, 28H), 0.85 (t, 3H, J = 6.6 Hz) |
| 26 | 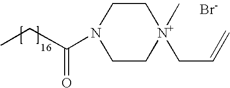 | 406(−Br) | 6.11-6.00 (m, 1H), 5.67 (s, 1H), 5.64 (d, 1H, J = 14.0 Hz), 4.09 (d, 2H, J = 3.6 Hz), 3.95-3.88 (m, 2H), 3.76 (t, 1H, J = 7.8 Hz), 3.67-3.61 (m, 1H), 3.41-3.26 (m, 4H), 3.07 (s, 3H), 2.34 (t, 2H, J = 7.2 Hz), 1.48 (t, 2H, J = 6.8 Hz), 1.3-1.2 (m, 28H), 0.85 (t, 3H, J = 6.6 Hz) |
| 27 | 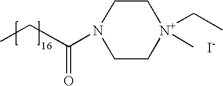 | 394(−I) | 3.97-3.81 (m, 2H), 3.78-3.68 (m, 2H), 3.51-3.41 (m, 2H), 3.39-3.30 (m, 4H), 3.05 (s, 3H), 2.33 (t, 2H, J = 7.4 Hz), 1.3- 1.2 (m, 31H), 0.85 (t, 3H, J = 6.6 Hz) |
| 28 | 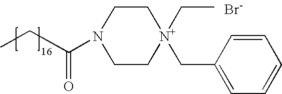 | 472(−Br) | 9.50 (s, 2H), 7.57-7.32 (m, 3H), 4.44 (s, 2H), 4.06 (d, 2H, J = 8.0 Hz), 3.47 (s, 2H), 3.15 (d, 2H, J = 4.0 Hz), 2.92 (t, 4H, J = 18.8 Hz), 2.35-2.31 (m, 2H), 1.47 (t, 2H, J = 6.0 Hz), 1.23-1.20 (m, 31H), 0.85 (t, 3H, J = 6.2 Hz) |
| 29 | 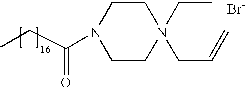 | 419(−Br) | 7.62-7.55 (m, 1H), 6.95-6.87 (m, 2H), 5.95-5.87 (m, 2H), 4.79-4.70 (m, 2H), 4.42-4.29 (m, 2H), 4.13-3.93 (m, 4H), 3.56-3.52 (m, 2H), 3.12-3.07 (m, 2H), 2.30 (t, 3H, J = 6.9 Hz), 1.39-1.24 (m, 30H), 0.85 (t, 3H, J = 6.6 Hz) |
| 30 | 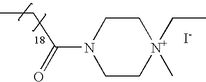 | 424(−I) | 3.81-3.46 (m, 4H), 3.41-3.25 (m, 4H), 3.12 (m, 2H), 3.04 (s, 3H), 2.32 (t, 2H, J = 7.3 Hz), 1.46 (m, 2H), 1.22 (m, 35H), 0.84 (t, 3H, J = 6.5 Hz) |
| 31 | 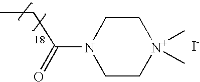 | 410(−I) | 3.78 (m, 4H), 3.39 (m, 4H), 3.14 (s, 6H), 2.33 (t, 2H, J = 7.6 Hz), 1.48 (m, 2H), 1.23 (m, 32H), 0.85 (t, 3H, 6.4 Hz.) |
| 32 | 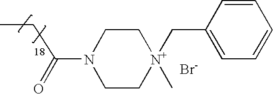 | 486(−Br) | 3.56-7.49 (m, 5H), 4.71 (S, 2H), 4.21- 3.24 (m, 8H), 3.03 (s, 3H), 2.39-2.30 (m, 2H), 1.38 (br m, 2H), 1.23 (br s, 32H), 0.85 (t, 3H, J = 6.8 Hz) |
| 33 | 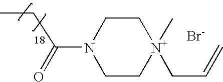 | 436(−Br) | 6.03-5.88 (m, 2H), 5.81 (br s, 1H) 4.70- 3.65 (m, 10H), 3.50 (s, 3H), 2.35 (t, 2H, J = 7.6 Hz), 1.59 (br m, 2H), 1.23 (br s, 36H), 0.88-0.83 (t, 3H, J = 6.7 Hz) |
| 34 | 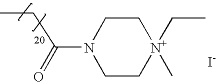 | 452(−I) | 3.95-3.55 (m, 4H), 3.47 (br m, 2H), 3.38 (br m, 4H), 3.06 (s, 3H), 2.34 (t, 2H, J = 7.2 Hz), 1.48 (br m, 2H), 1.23 (br s, 39H), 0.85 (t, 3H, J = 6.0 Hz) |
| 35 | 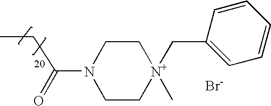 | 514(−Br) | 7.55 (br m, 5H), 4.67 (s, 2H), 4.18-3.71 (m, 4H), 3.47 (m, 4H), 3.03 (s, 3H), 2.34 (br s, 2H), 1.48 (br m, 2H), 1.24 (br s, 36H), 0.86 (br s, 3H) |
| 36 | 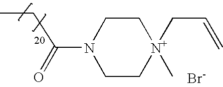 | 464(−Br) | 6.09-5.98 (m, 1H), 5.67-5.62 (m, 2H), 4.12 (d, 2H, J = 7.6 Hz), 3.95-3.62 (m, 4H), 3.37 (br m, 4H), 3.08 (s, 3H), 2.34 (t, 2H, J = 7.6 Hz), 1.47 (br m, 2H), 1.23 (br s, 36H), 0.86 (t, 3H, J = 6.0 Hz) |
| 37 | 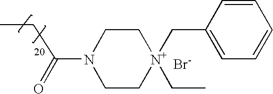 | 481(−Br) | 7.51 (br m, 5H), 4.67 (s, 2H), 4.11-3.55 (m, 4H), 3.46-3.40 (m, 6H), 2.39-2.31 (m, 2H), 1.49 (br m, 2H), 1.36 (t, 3H, J = 7.2 Hz), 1.23 (br s, 36H), 0.84 (t, 3H, J = 6.4 Hz) |
| 38 | 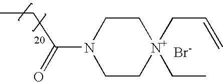 | 478(−Br) | 6.01-5.98 (m, 1H), 5.72-5.65 (m, 2H), 4.08 (d, 2H, J = 7.6 Hz), 3.79 (br m, 4H), 3.43 (br m, 5H), 2.34 (t, 2H, J = 7.2 Hz), 1.47 (br m, 2H), 1.23 (br s, 39H), 0.86 (t, 3H, J = 5.6 Hz) |
| 39 | 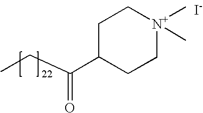 | 466(−I) | 3.69 (br m, 4H), 3.26 (br m, 4H), 3.06 (s, 6H), 2.25 (t, 2H, J = 7.3 Hz), 1.39 (br m, 2H), 1.14 (br s, 40H), 0.76 (t, 3H, J = 6.0 Hz) |
| 40 | 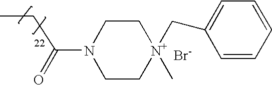 | 542(−Br) | 7.52 (br m, 5H), 4.63-3.70 (m, 6H), 3.32 (br m, 4H), 3.00 (s, 3H), 2.28 (br m, 2H), 1.47 (br m, 2H), 1.22 (br s, 40H), 0.83 (t, 3H, J = 6.3 Hz) |
| 41 | 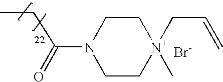 | 492(−Br) | 6.10-5.85 (m, 2H), 5.80-5.70 (m, 1H), 4.81-3.74 (m, 10H), 3.51 (s, 3H), 2.37 (br m, 2H), 1.62 (br m, 2H), 1.23 (br s, 40H), 0.86 (t, 3H, J = 6.7 Hz) |
| 42 | 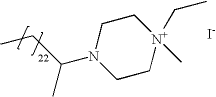 | 480(−I) | 3.41-3.89 (m, 4H), 3.30 (br m, 8H), 3.12 (s, 3H), 3.04 (br m, 2H), 2.32 (t, 2H, J = 7.4 Hz), 1.46 (br m, 2H), 1.21(br s, 43H), 0,83 (t, 3H, J = 6.5 Hz) |
| 43 | 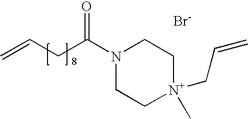 | 306(−Br) | 6.11-5.99 (m, 1H), 5.83-5.63 (m, 2H), 5.02-4.91 (m, 2H), 4.13 (d, 2H, J = 3.6 Hz), 3.90-3.76 (br m, 4H), 3.39-3.30 (br m, 4H), 3.08 (s, 3H), 2.34 (t, 2H, J = 7.2 Hz), 2.01-1.97 (m, 2H), 1.48-1.46 (br m, 2H), 1.33-1.17 (br m, 10H) |
| 44 | 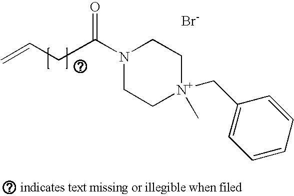 | 258(−Br) | 7.58-7.48 (m, 5H), 5.85-5.72 (m, 1H), 5.02-4.91 (m, 2H), 4.70 (s, 2H), 4.20- 3.98 (m, 2H), 3.75-3.68 (m, 2H), 3.48- 3.34 (m, 4H), 3.00 (s, 3H), 2.50-2.31 (m, 2H), 2.04-1.97 (m, 2H), 1.48-1.46 (m, 2H), 1.33-1.16 (m, 10H) |
| 45 | 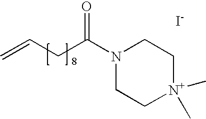 | 281(−I) | 5.86-5.73 (m, 1H), 5.55-5.48 (m, 2H), 3.93 (br s, 4H), 3.52-3.48 (br m, 4H), 3.33-3.08 (m, 6H), 3.01-2.42 (m, 2H), 2.05-2.00 (m, 2H), 1.63-1.60 (m, 2H), 1.33 (s, 10H) |
| 46 | 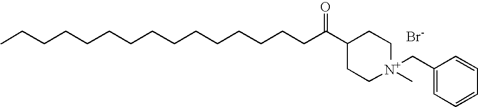 | | 7.67-7.43 (m, 5H), 5.28-5.15 (m, 2H), 4.00 (br s, 2H), 3.80 (br m, 2H), 3.55 (br m, 4H), 3.01 (s, 3H), 2.35-2.30 (m, 2H), 1.55 (br m, 2H), 1.31-1.16 (m, 24H), 0.95-0.84 (m, 3H) |
| 47 | 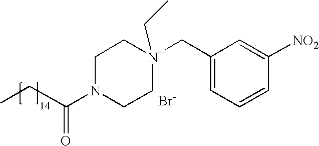 | 489(−Br) | 7.98-7.39 (m, 4H), 4.68 (s, 2H), 4.41-3.29 (m, 10H), 2.34 (br m, 2H), 1.44 (br m, 2H), 1.39-1.36 (m, 3H), 1.24 (br s, 24H), 0.83 (t, 3H, J = 6.7 Hz) |
| 48 | 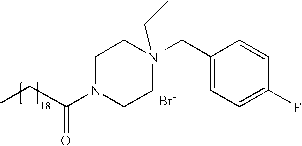 | 490(−Br) | 7.56-7.21 (m, 4H), 4.57 (s, 2H), 4.31-3.29 (m, 10H), 2.34 (br m, 2H), 1.46 (br m, 2H), 1.41-1.34 (m, 3H), 1.26 (br s, 28H), 0.89-0.85 (t, 3H, J = 6.8 Hz) |
| 49 | 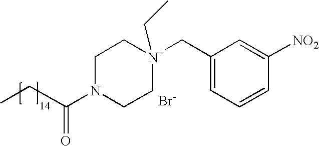 | 517(−Br) | 7.40-7.29 (m, 4H), 4.67 (s, 2H), 4.21- 3.28 (m, 10H), 2.36 (m, 2H), 1.46-1.43 (m, 2H), 1.40-1.36 (t, 3H, J = 7.2 Hz), 1.22 (br s, 28H), 0.85 (t, 3H, J = 6.6 Hz) |
| 50 | 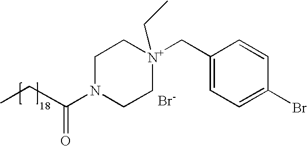 | 551(−Br) | 7.54-7.23 (m, 4H), 4.64 (s, 2H), 4.22- 3.27 (m, 10H), 2.35 (br m, 2H), 1.46-1.42 (m, 2H), 1.37 (br m, 3H), 1.24 (br s, 28H), 0.86 (t, 3H, J = 6.6 Hz) |
| 51 | 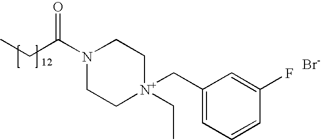 | 434(−Br) | 7.80-7.66 (m, 2H), 7.13 (t, 2H, J = 8.4 Hz), 5.27-5.10 (m, 2H), 4.54-4.34 (m, 1H), 4.16-3.98 (m, 1H), 3.94-3.46 (m, 8H), 2.38-2.22 (m, 2H), 1.55 (t, 5H, J = 7.2 Hz), 1.32-1.12 (m, 20H), 0.88 (t, 3H, J = 6.6 Hz) |
| 52 | 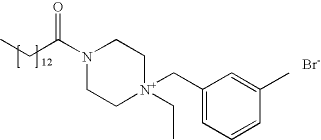 | 429(−Br) | 7.43-7.28 (m, 5H), 5.14-4.94 (m, 2H), 4.48-4.32 (m, 1H), 4.12-4.00 (m, 2H), 3.94-3.80 (m, 4H), 3.64-3.42 (m, 3H), 2.39 (s, 3H), 2.34-2.28 (m, 2H), 1.55 (t, 3H, J = 6.9 Hz), 1.34-1.20 (m, 22H), 0.88 (t, 3H, J = 6.6 Hz) |
| 53 | 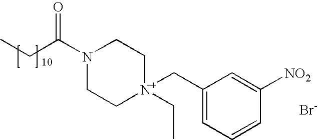 | 432(−Br) | 8.67-8.63 (m, 1H), 8.31 (t, 2H, J = 7.5 Hz), 7.70 (t, 1H, J = 7.8 Hz), 5.54-5.38 (m, 2H), 4.59-4.45 (m, 1H), 4.15-3.40 (m, 9H), 2.34-2.26 (m, 2H), 1.64 (t, 3H, J = 6.9 Hz), 1.55-1.49 (m, 2H), 1.32-1.16 (m, 16H), 0.87 (t, 3H, J = 6.6 Hz) |
| 54 | 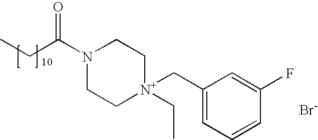 | 405(−Br) | 7.72-7.67 (m, 2H), 7.16 (t, 2H, J = 8.4 Hz), 5.27-5.14 (m, 2H) 4.67-4.50 (m, 1H, 4.15-3.43 (m, 9H), 2.41-2.25 (m, 2H), 1.58-1.52 (m, 3H), 1.32-1.22 (m, 18H), 0.88 (t, 3H, J = 6.6 Hz) |
| 55 | 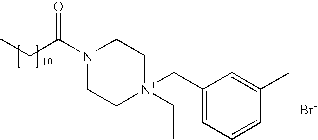 | 401(−Br) | 7.42-7.29 (m, 4H), 5.13-5.00 (m, 2H), 4.46-4.36 (m, 1H), 4.14-4.00 (m, 2H), 3.94-3.76 (m, 4H), 3.62-3.40 (m, 3H), 2.39 (s, 3H), 2.30 (t, 2H, J = 7.2 Hz), 1.55 (t, 3H, J = 7.2 Hz), 1.30-1.16 (m, 16H), 0.68 (t, 3H J = 6.6 Hz) |
| 56 | 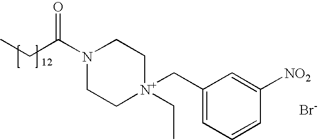 | 480(−Br) | 8.60 (s, 1H), 8.35-8.29 (m, 2H), 7.70 (t, 1H, J = 7.5 Hz), 5.50-5.40 (m, 2H), 4.15- 3.45 (m, 10H), 2.35-2.20 (m, 2H), 1.63 (t, 2H, J = 6.8 Hz), 1.56-1.50 (m, 2H), 1.32- 1.20 (m, 20H), 0.87 (t, 3H, J = 6.6 Hz) |
| 57 | 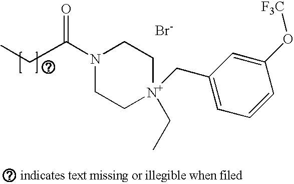 | 556(−Br) | 7.40-7.29 (m, 4H), 4.67 (s, 2H), 4.21- 3.28 (m, 10H), 2.36 (br m, 2H), 1.46-1.43 (m, 2H), 1.38 (t, 3H, J = 7.2 Hz), 1.22 (br s, 28H), 0.85 (t, 3H, J = 6.6 Hz) |
| 58 | 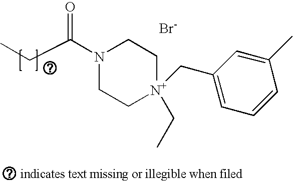 | 486(−Br) | 7.42-7.31 (m, 4H), 4.63 (s, 2H), 4.14-3.27 (m, 10H), 2.38-2.27 (m, 5H), 1.48-1.45 (m, 2H), 1.37 (t, 3H, J = 7.2 Hz), 1.23 (br s, 28H), 0.85 (t, 3H, J = 6.8 Hz) |

### Example 59

### Preparation of 1-ethyl-1-methyl-4-(octadecane-1-sulfonyl)piperazin-1-ium iodide according to Reaction Scheme 2

### 1. Preparation of 1-methyl-4-(octadecane-1-sulfonyl)piperazine

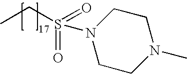

Oxalyl chloride (2.8□ (2.0 M in methylene chloride), 5.6 mmol) and N,N-dimethylformamide (0.3□, 0.004 mmol) were added to a 0.6 M methylene chloride solution of octadecano-1-sulfonic acid (2 g, 5.6 mmol) under stirring, and heated for 4 hours. The produced mixture was cooled to room temperature, and then filtered. Then, methylpiperazine (0.9□, 8.4 mmol) was added to the filtrate at 0° C. under stirring for 2 hours. The produced mixture was diluted with methylene chloride, and then washed with saturated ammonium chloride. The organic layer was washed with the saturated brine solution, dried over magnesium sulfate, and distilled off under reduced pressure. The resulting primary compound was purified by a silica gel column chromatography (eluent: 5% methanol/chloroform) to obtain the target compound in 30% yield (702 mg).

¹H-NMR (300 MHz, CDCl₃) δ 3.32-3.30 (m, 4H), 2.92-2.87 (m, 2H), 2.51-2.48 (m, 4H), 2.34 (s, 3H), 1.88-1.75 (m, 2H), 1.42-1.26 (m, 30H), 0.88 (t, 3H, J=6.6 Hz)

### 2. Preparation of 1-ethyl-1-methyl-4-(octadecane-1-sulfonyl)piperazin-1-ium iodide

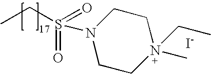

Iodoethane (0.07□, 0.87 mmol) was added to a 0.1 M acetonitrile solution of the compound (150 mg, 0.36 mmol) obtained in the step 1 under stirring, and heated for 6 hours. The produced mixture was sufficiently cooled to 0° C., and then filtered with eluding ethyl acetate solvent to obtain the target compound in 83% yield (168 mg).

¹H-NMR (300 MHz CDCl₃) δ3.94-3.81 (m, 6H), 3.81-3.77 (m, 2H), 3.64-3.56 (m, 2H), 3.52 (s, 3H), 3.19 (t, 2H, J=7.9 Hz), 1.81-1.75 (m, 2H), 1.49-1.44 (m, 5H), 1.23 (br s, 28H), 0.86 (t, 3H, J=6.7 Hz)

### Examples 60 to 115

The compounds of Examples 60 to 115 were prepared in a similar manner to the preparation process that is described in Example 59.

The physical properties of the compounds are shown in Table 2.

TABLE 2

| | | MS data | |
|---|---|---|---|
| | | (M⁺H), | |
| Example | Chemical structure | (g/mol) | ¹H NMR spectrum data |
| 60 | 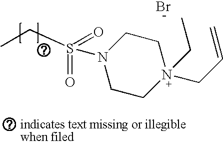 | 332(−Br) | 6.02-5.98 (m, 1H), 5.63 (br m, 2H), 4.14-4.10 (m, 2H), 3.58-3.56 (m, 4H), 3.52-3.44 (m, 6H), 3.17-3.14 (t, 2H, J = 7.7 Hz), 1.38-1.37 (m, 2H), 1.25-1.21 (m, 13H), 0.86 (t, 3H, J = 6.6 Hz) |
| 61 | 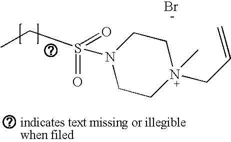 | 318(−Br) | 6.10-5.99 (m, 1H), 5.75-5.65 (m, 2H), 4.16-4.07 (m, 2H), 3.60-3.55 (m, 4H), 3.48-3.47 (m, 4H), 3.17-3.12 (m, 2H), 3.09 (s, 3H), 1.71-1.66 (m, 2H), 1.26 (br s, 10H), 0.86 (t, 3H, J = 5.7 Hz) |
| 62 | 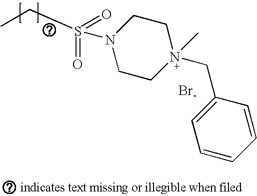 | 368(−Br) | 7.60-7.53 (m, 5H), 4.74 (s, 2H), 3.55-3.52(m, 4H), 3.23-3.22 (m, 4H), 3.18-3.13 (m, 2H), 3.05 (s, 3H), 1.77-1.64 (m, 2H), 1.26 (br s, 10H), 0.86 (t, 3H, J = 6.3 Hz) |
| 63 | 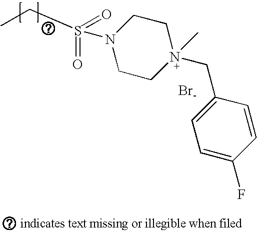 | 386(−Br) | 7.67-7.62 (m, 2H), 7.41-7.35 (m, 2H), 4.73 (s, 2H), 3.73-3.69 (m, 2H), 3.54-3.52 (m, 4H), 3.21 (br, 2H), 3.18-3.13 (t, 2H, J = 7.8 Hz), 1.71-1.64 (m, 2H), 1.26 (br s, 10H), 0.87 (t, 3H, J = 6.6 Hz) |
| 64 | 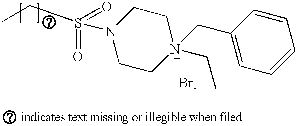 | 424(−Br) | 7.59-7.50 (m, 5H), 4.67 (s, 2H), 3.86-3.81 (m, 2H), 3.67-3.44 (m, 8H), 3.10 (t, 2H, J = 7.9 Hz). 1.81-1.76 (m, 2H), 1.52-1.29 (m, 19H), 0.89 (t, 3H, J = 6.6 Hz) |
| 65 | 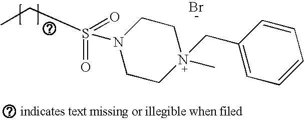 | 410(−Br) | 7.60-7.55 (m, 5H), 4.57 (s, 2H), 3.88-3.84 (m, 2H), 3.64-3.57 (m, 4H), 3.53-3.49 (m, 2H), 3.17-3.12 (m, 5H), 1.82-1.77 (m, 2H), 1.29 (br s, 16H), 0.89 (t, 3H, J = 6.3 Hz) |
| 66 | 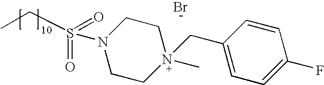 | 428(−Br) | 7.66-7.51 (m, 2H), 7.33-7.26 (m, 2H), 4.68 (s, 2H), 3.88-3.84 (m, 2H), 3.63-3.48 (m, 6H), 3.17-3.10 (m, 5H), 1.82-1.77 (m, 2H), 1.30 (br s, 16H), 0.89 (t, 3H, J = 6.3 Hz) |
| 67 | 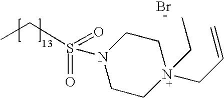 | 416(−Br) | 6.02-5.96 (m, 1H), 5.74-5.63 (m, 2H), 4.13 (d, 2H, J = 7.0 Hz), 3.58-3.52 (m, 4H), 3.48-3.39 (m, 6H), 3.17-3.13 (m, 2H), 1.72-1.64 (m, 2H), 1.24 (br s, 25H), 0.85 (t, 3H, J = 6.8 Hz) |
| 68 | 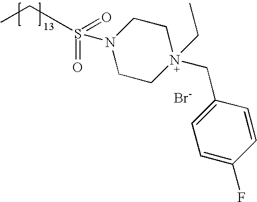 | 484(−Br) | 7.64-7.59 (m, 2H), 7.41-7.35 (m, 2H), 4.69 (s, 2H), 3.66-3.51 (m, 8H), 3.21-3.20 (m, 2H), 3.14 (t, 2H, J = 7.5 Hz), 1.67 (br m, 2H), 1.24 (br s, 25H), 0.86 (t, 3H, J = 6.9 Hz) |
| 69 | 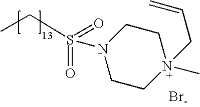 | 402(−Br) | 6.12-6.01 (m, 1H), 5.70-5.66 (m, 2H), 4.15 (d, 2H J = 7.3 Hz), 3.64-3.47 (m, 8H), 3.15 (t, 2H J = 7.7 Hz), 3.10 (s, 3H), 1.71-1.64 (m, 2H), 1.38-1.24 (m, 22H), 0.85 (t, 3H, J = 6.2 Hz) |
| 70 | 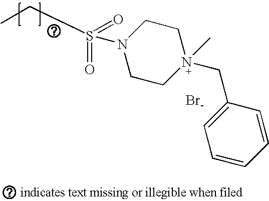 | 451(−Br) | 7.56-7.55 (m, 5H), 4.71 (s, 2H), 3.73-3.69 (m, 2H), 3.55-3.52 (m, 6H), 3.15 (t, 2H, J = 7.8 Hz), 1.71-1.63 (m, 2H), 1.24 (br s, 22H), 0.85 (t, 3H, J = 6.6 Hz) |
| 71 | 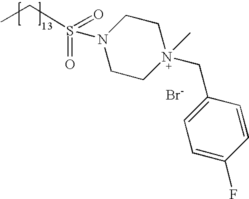 | 470(−Br) | 7.65-7.61 (m, 2H), 7.41-7.36 (m, 2H), 4.70 (s, 2H), 3.71-3.68 (m, 2H), 3.54-3.50 (m, 4H), 3.43-3.41 (m, 2H), 3.14 (t, 2H, J = 7.9 Hz), 3.02 (s, 3H) 1.70-1.66 (m, 2H), 1.24 (br s, 22H), 0.86 (t, 3H, J = 6.8 Hz) |
| 72 | 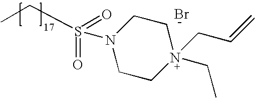 | 472(−Br) | 6.03-5.82 (m, 2H), 5.77 (m, 1H), 4.52 (d, 2H, J = 6.4 Hz), 3.98-3.57 (m, 10H), 3.24 (t, 2H, J = 7.9 Hz), 1.86-1.72 (m, 2H), 1.48-1.39 (m, 3H), 1.23 (br, 30H), 0.85 (t, 3H, J = 6.6 Hz) |
| 73 | 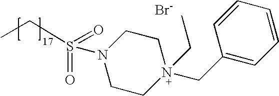 | 522(−Br) | 7.54-7.52 (m, 5H), 4.68 (s, 2H), 3.69-3.64 (m, 2H), 3.50-3.48 (m, 4H), 3.19-3.18 (m, 4H), 3.14-3.11 (m, 2H), 1.67-1.60 (m, 2H), 1.24 (br s, 33H), 0.85 (t, 3H, J = 6.0 Hz) |
| 74 | 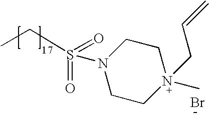 | 458(−Br) | 5.98-5.95 (m, 2H), 5.80 (t, 1H, J = 5.6 Hz), 4.64-3.82 (m, 6H), 3.70-3.69 (m, 4H), 3.54 (s, 3H), 3.19 (t, 2H, J = 7.8 Hz), 1.86-1.76 (m, 2H), 1.46-1.41 (m, 2H), 1.23 (br s, 28H), 0.86 (t, 3H, J = 6.6 Hz) |
| 75 | 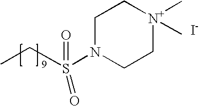 | | 3.87 (d, 4H, J = 2.4 Hz), 3.80 (d, 4H, J = 2.3 Hz), 3.68 (s, 6H), 3.19 (t, 2H, J = 7.8 Hz), 1.87-1.77 (m, 2H), 1.50-1.23 (m, 14H), 0.88 (t, 3H, J = 6.6 Hz) |
| 76 | 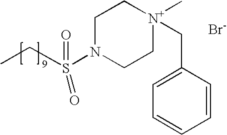 | | 7.67 (d, 2H, J = 3.3 Hz), 7.53-7.42 (m, 3H), 5.36 (s, 2H), 4.10-3.92 (m, 2H), 3.84-3.63 (m, 6H), 3.50 (s, 3H), 3.21 (t, 2H J = 7.8 Hz), 1.89-1.77 (m, 2H), 1.50-1.22 (m, 14H), 0.87 (t, 3H, J = 6.6 Hz) |
| 77 | 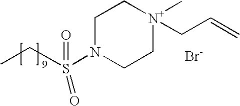 | | 6.00 (d, 2H, J = 1.6 Hz), 5.83-5.79 (m, 1H), 4.67 (d, 2H, J = 2.5 Hz), 4.20-3.84 (m, 4H), 3.80-3.68 (m, 4H), 3.56 (s, 3H), 3.22 (t, 2H, J = 7.8 Hz), 1.88-1.77 (m, 2H), 1.51-1.22 (m, 14H), 0.88 (t, 3H, J = 6.6 Hz) |
| 78 | 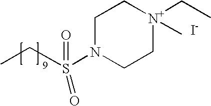 | | 3.96-3.80 (m, 8H), 3.68-3.60 (m, 2H), 3.54 (s, 3H), 3.21 (t, 2H, J = 7.8 Hz), 1.88-1.77 (m, 2H), 1.48 (t, 3H, J = 6.6 Hz), 1.23 (br m, 14H), 0.88 (t, 3H, J = 6.0 Hz) |
| 79 | 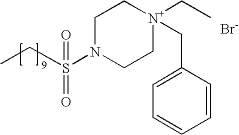 | | 7.55 (s, 5H), 4.73 (s, 2H), 3.70 (d, 2H, J = 5.6 Hz), 3.56-3.40 (m, 8H), 3.20-3.13 (m, 2H), 1.73-1.63 (m, 2H), 1.37 (t, 3H, J = 7.0 Hz), 1.22 (br m, 14H), 0.86 (t, 3H, J = 6.3 Hz) |
| 80 | 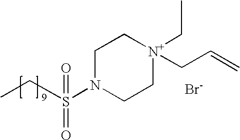 | | 6.07-5.86 (m, 2H), 5.80 (d, 1H, J = 4.6 Hz), 4.58 (d, 2H, J = 3.0 Hz), 4.08-3.98 (m, 2H), 3.94-3.75 (m, 6H), 3.68-3.60 (m, 2H), 3.28 (t, 2H, J = 7.8 Hz), 1.88-1.78 (m, 2H), 1.46 (t, 3H, J = 6.0 Hz), 1.36-1.22 (m, 14H), 0.88 (t, 3H, J = 6.0 Hz) |
| 81 | 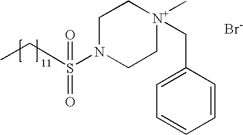 | | 7.67 (d, 2H, J = 3.0 Hz), 7.51-7.42 (m, 3H), 5.36 (s, 2H), 3.96 (d, 2H, J = 6.0 Hz), 3.83-3.64 (m, 6H), 3.50 (s, 3H), 3.21 (t, 2H, J = 7.8 Hz), 1.87-1.77 (m, 2H), 1.51-1.21 (m, 18H), 0.88 (t, 3H, J = 6.0 Hz) |
| 82 | 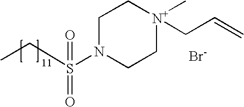 | | 6.15-6.03 (m, 1H), 5.79-5.69 (m, 2H), 4.18 (d, 2H, J = 3.6 Hz), 3.64-3.58 (m, 2H), 3.51 (d, 4H, J = 3.0 Hz), 3.19 (t, 2H, J = 4.8 Hz), 3.13 (s, 3H), 1.78-1.67 (m, 2H), 1.46-1.17 (m, 18H), 0.89 (t, 3H, J = 6.0 Hz) |
| 83 | 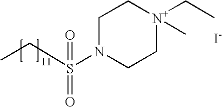 | | 3.77-3.51 (m, 10H), 3.23-3.16 (m, 6H), 1.84-1.75 (m, 2H), 1.58-1.21 (m, 21H), 0.89 (t, 3H, J = 5.1 Hz) |
| 84 | 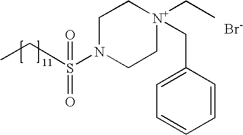 | | 7.54 (s, 5H), 4.69 (s, 2H), 3.68 (d, 2H, J = 6.0 Hz), 3.60-3.32 (m, 8H). 3.15 (t, 2H, J = 6.0 Hz) 1.69-1.65 (m, 2H), 1.37 (t, 3H, J = 7.2 Hz), 1.29-1.23 (m, 18H), 0.85 (t, 3H, J = 9.0 Hz) |
| 85 | 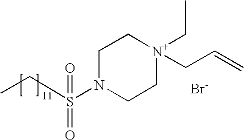 | | 6.08-5.94 (m, 1H), 5.73-5.63 (m, 2H), 4.11 (d, 2H, J = 3.0 Hz), 3.57-3.55 (m, 4H), 3.48-3.36 (m, 6H), 3.15 (t, 2H, J = 6.0 Hz), 1.73-1.63 (m, 2H). 1.38-1.17 (m, 21H), 0.85 (t, 3H, J = 6.0 Hz) |
| 86 | 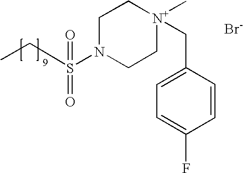 | | 7.65-7.61 (m, 3H), 7.38 (t, 2H, J = 8.0 Hz), 4.70 (s, 2H), 3.70 (d, 2H, J = 6.0 Hz), 3.54-3.47 (m, 4H), 3.42 (d, 2H, J = 6.0 Hz), 3.14 (t, 2H, J = 8.0 Hz), 3.02 (s, 3H), 1.70-1.64 (m, 2H), 1.40 (m, 14H), 0.85 (t, 3H, J = 8.0 Hz) |
| 87 | 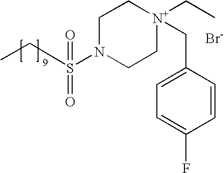 | | 7.60 (t, 2H, J = 5.6 Hz), 7.38 (t, 2H, J = 8.0 Hz), 4.67 (s, 2H), 3.67 (d, 2H, J = 6.6 Hz), 3.53-3.39 (m, 8H), 3.13 (t, 2H, J = 8.0 Hz), 1.69-1.63 (m, 2H), 1.36 (t, 3H, J = 8.0 Hz), 1.30-1.20 (m, 14H), 0.86 (t, 3H, J = 6.0 Hz) |
| 88 | 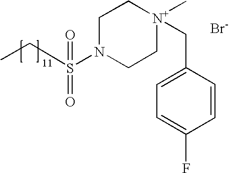 | | 7.65-7.61 (m, 2H), 7.38 (t, 2H, J = 8.0 Hz), 4.71 (s, 2H), 3.70 (d, 2H, J = 6.2 Hz), 3.56-3.47 (m, 4H), 3.42 (d, 2H, J = 5.6 Hz), 3.14 (t, 2H, J = 8.0 Hz), 3.03 (s, 3H), 1.69-1.66 (m, 2H), 1.37-1.22 (m, 18H), 0.85 (t. 3H, J = 6.4 Hz) |
| 89 | 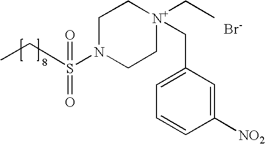 | | 8.47 (s, 1H), 8.41 (d, 1H, J = 4.0 Hz), 8.02 (d, 1H, J = 4.0 Hz), 7.83 (t, 1H, J = 8.0 Hz), 4.87 (s, 2H), 3.72 (d, 2H, J = 6.6 Hz), 3.60-3.40 (m, 6H), 3.13-3.09 (m, 4H), 1.70-1.60 (m, 2H), 1.50-1.10 (m, 15H), 0.86 (t, 3H, J = 6.2 Hz) |
| 90 | 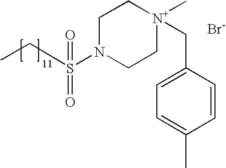 | | 7.44 (d, 2H, J = 4.0 Hz), 7.33 (d, 2H, J = 4.0 Hz), 4.65 (s, 2H), 3.69 (d. 2H, J = 5.6 Hz), 3.53 (d, 4H, J = 4.0 Hz), 3.42-3.40 (m, 3H), 3.17-3.12 (m, 2H), 3.00 (s, 3H), 2.36 (s, 3H), 1.70-1.63 (m, 2H), 1.40-1.20 (m, 18H), 1.85 (t, 3H, J = 6.4 Hz) |
| 91 | 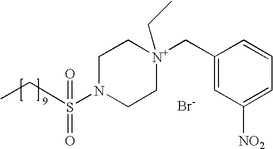 | | 8.42 (s, 1H), 8.39 (s, 1H), 7.98 (d, 1H, J = 3.9 Hz), 7.83 (t, 1H, J = 7.6 Hz), 4.83 (s, 2H). 3.72-3.64 (m, 2H), 3.56-3.41 (m, 8H), 3.13 (t, 2H, J = 7.5 Hz), 1.73-1.60 (m, 2H), 1.40 (t, 3H, J = 7.0 Hz), 1.33-1.19 (m, 14H), 0.86 (t, 3H, J = 6.6 Hz) |
| 92 | 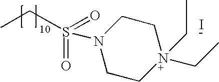 | 362(−I) | 3.51-3.45 (m, 12H), 3.14 (t, 3H, J = 7.7 Hz), 1.68 (br m, 2H), 1.25-1.17 (m, 22H), 0.86 (t, 3H, J = 7.0 Hz) |
| 93 | 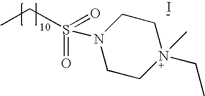 | 348(−I) | 3.55-3.47 (m, 10H), 3.14 (t, 2H, J = 7.8 Hz), 3.07 (s, 3H), 1.68-1.66 (m, 2H), 1.38-1.25 (m, 19H), 0.86 (t, 3H, J = 6.7 Hz) |
| 94 | 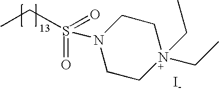 | 404(−I) | 3.52-3.43 (m, 12H), 3.14 (t, 2H, J = 7.8 Hz), 1.68-1.66 (m, 2H), 1.38-1.24 (m, 28H), 0.86 (t, 3H, J = 6.9 Hz) |
| 95 | 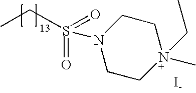 | 390(−I) | 3.56-3.43 (m, 10H), 3.14 (t, 2H, J = 7.8 Hz), 3.07 (s, 3H), 1.68-1.66 (m, 2H), 1.38-1.17 (m, 25H), 0.86 (t, 3H, J = 6.0 Hz) |
| 96 | 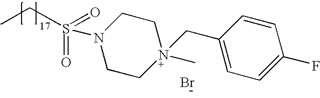 | 526(−Br) | 7.65-7.61 (m, 2H), 7.41-7.35 (m, 2H), 4.70 (s, 2H), 3.72-3.40 (m, 8H), 3.02 (s, 3H), 1.68-1.66 (m, 2H), 1.23 (br s, 30H), 0.85 (t, 3H, J = 5.3 Hz) |
| 97 | 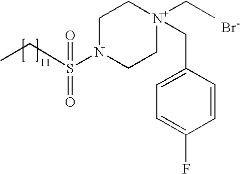 | | 7.62-7.57 (m, 2H), 7.37 (t, 2H, J = 8.4 Hz), 4.67 (s, 2H), 3.68 (d, 2H, J = 3.0 Hz), 3.51-3.36 (m, 8H), 3.13 (t, 2H, J = 7.5 Hz), 1.67-1.62 (m, 2H), 1.40-1.22 (m, 21H), 0.85 (t, 3H, J = 6.6 Hz) |
| 98 | 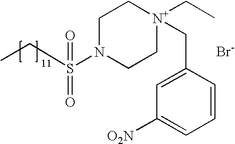 | | 8.40 (d, 2H, J = 6.0 Hz), 7.99 (d, 1H, 3.0 Hz), 7.83 (t, 1H, J = 8.4 Hz), 4.85 (s, 2H), 3.67 (d, 2H, J = 6.0 Hz), 3.56-3.44 (m, 8H), 3.13 (t, 2H, J = 7.5 Hz), 1.67-1.61 (m, 2H), 1.40 (t, 3H, J = 6.9 Hz), 1.30-1.22 (m, 18H), 0.85 (t, 3H, J = 6.6 Hz) |
| 99 | 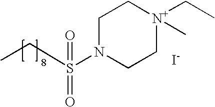 | | 3.55-3.42 (m, 10H), 3.14 (t, 2H, J = 7.8 Hz), 3.07 (s, 3H), 1.71-1.63 (m, 2H), 1.38-1.25 (m, 15H), 0.85 (t, 3H, J = 6.6 Hz) |
| 100 | 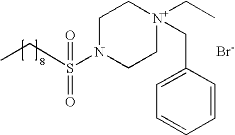 | | 7.54 (s, 5H), 4.70 (s, 2H), 3.69 (d, 2H, J = 6.4 Hz), 3.14 (t, 2H, J = 7.6 Hz), 1.68-1.63 (m, 2H), 1.37 (t, 3H, J = 6.6 Hz), 1.36-1.24 (m, 12H), 0.85 (t, 3H, J = 6.6 Hz) |
| 101 | 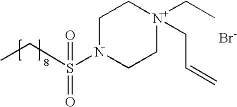 | | 6.07-5.94 (m, 1H), 5.74-5.62 (m, 2H), 4.12 (d, 2H, J = 3.0 Hz), 3.58-3.56 (m, 4H), 3.50-3.41(m, 6H), 3.15 (t, 2H, J = 7.8 Hz), 1.69-1.62 (m, 2H), 1.39-1.21 (m, 15H), 0.85 (t, 3H, J = 6.6 Hz) |
| 102 | 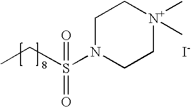 | | 3.55-3.48 (m, 8H), 3.18-3.11 (m, 6H), 1.69-1.62 (m, 2H), 1.40-1.24 (m, 12H), 0.85 (t, 3H, J = 6.6 Hz) |
| 103 | 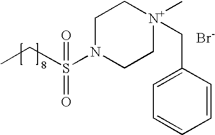 | | 7.55 (s, 5H), 4.70 (s, 2H), 3.70 (d, 2H, J = 6.4 Hz), 3.54-3.40 (m, 6H), 3.14 (t, 2H, J = 8.4 Hz), 3.03 (s, 3H), 1.73-1.62 (m, 2H), 1.39-1.23 (m, 12H), 0.86 (t, 3H, J = 6.6 Hz) |
| 104 | 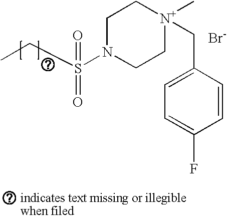 | | 7.66-7.60 (m, 2H), 7.38 (t, 2H, J = 8.7 Hz), 4.69 (s, 2H), 3.70 (d, 2H, J = 5.4 Hz), 3.54-3.49 (m, 4H), 3.44-3.40(m, 2H), 3.14 (t, 2H, J = 7.8 Hz), 3.02 (s, 3H), 1.70-1.66 (m, 2H), 1.36-1.24 (m, 12H), 0.85 (t, 3H, J = 6.6 Hz) |
| 105 | 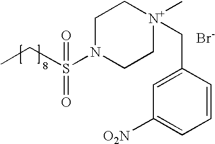 | | 8.46-8.37 (m, 2H), 8.01 (d, 1H, J = 3.8 Hz), 7.82 (t, 1H, J = 7.2 Hz), 4.87 (s, 2H), 3.75-3.43 (m, 8H), 3.14 (t, 2H, J = 7.8 Hz), 3.08 (s, 3H), 1.69-1.67 (m, 2H), 1.38-1.23 (m, 14H), 0.84 (t, 3H, J = 6.9 Hz) |
| 106 | 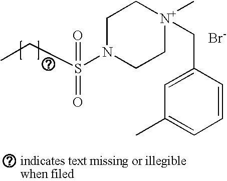 | | 7.42-7.35 (m, 4H), 4.65 (s, 2H), 3.69 (d, 2H, J = 5.4 Hz), 3.54-3.40 (m, 6H), 3.13 (t, 2H), J = 7.5 Hz), 3.02 (s, 3H), 2.36 (s, 3H), 1.69-1.64 (m, 2H), 1.39-1.14 (m, 14H), 0.84 (t, 3H, J = 6.6 Hz) |
| 107 | 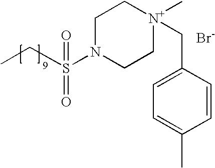 | | 7.44 (d, 2H, J = 4.0 Hz), 7.33 (d, 2H, J = 3.9 Hz), 4.64 (s, 2H), 3.69-3.34 (m, 8H), 3.13 (t, 2H, J = 7.8 Hz), 3.00 (s, 3H), 2.36 (s, 3H), 1.69-1.63 (m, 2H), 1.40-1.23 (m, 14H), 0.85 (t, 3H, J = 6.6 Hz) |
| 108 | 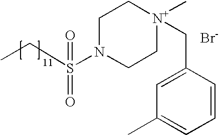 | | 7.45-7.37 (m, 4H), 4.66 (s, 2H), 3.70 (d, 2H, J = 6.0 Hz), 3.56-3.40 (m, 6H), 3.14 (t, 2H, J = 7.8 Hz), 3.03 (s, 3H), 2.37 (s, 3H), 1.72-1.63 (m, 2H), 1.40-1.23 (m, 18H), 0.85 (t, 3H, J = 6.6 Hz) |
| 109 | 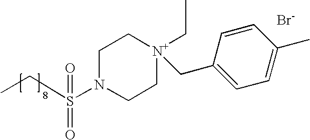 | | 7.42 (d, 2H, J = 4.0 Hz), 7.33 (d, 2H, J = 3.9 Hz), 4.63 (s, 2H), 3.67 (d, 2H, J = 6.7 Hz), 3.55-3.34 (m, 8H), 3.13 (t, 2H, J = 7.8 Hz), 2.36 (s, 3H), 1.70-1.62 (m, 2H), 1.36 (t, 3H, J = 6.6 Hz), 1.31-1.23 (m, 14H), 0.85 (t, 3H, J = 6.6 Hz) |
| 110 | 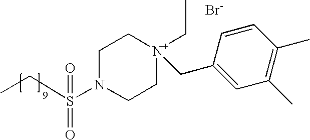 | | 7.45-7.32 (m, 4H), 4.64 (s, 2H), 3.68 (d, 2H, J = 6.8 Hz), 3.56-3.36 (m, 8H), 3.14 (t, 2H, J = 7.8 Hz), 2.37 (s, 3H), 1.72-1.62 (m, 2H), 1.36 (t, 3H, J = 6.9 Hz), 1.24 (br m, 14H), 0.85 (t, 3H, J = 6.7 Hz) |
| 111 | 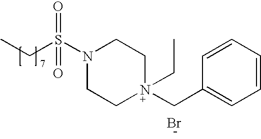 | 382(−Br) | 7.56-7.50 (m, 5H), 4.68 (s, 2H), 3.68-3.35 (m, 10H), 3.13 (t, 2H, J = 7.8 Hz), 1.67 (br m, 2H), 1.36 (t, 5H, J = 7.1 Hz), 1.25 (br s, 8H), 0.85 (t, 3H, J = 6.8 Hz) |
| 112 | 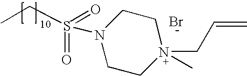 | 360(−Br) | 5.97 (d, 2H, J = 4.9 Hz), 5.83-5.79 (m, 1H), 4.60 (d, 2H, J = 7.7 Hz), 3.96-3.69 (m, 8H), 3.53 (s, 3H), 3.19 (t, 2H), J = 7.7 Hz), 1.76 (br m, 2H) 1.23 (br s, 16H), 0.88 (t, 3H, J = 6.6 Hz) |
| 113 | 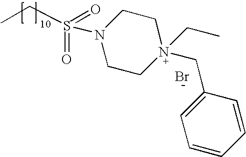 | 466(−Br) | 7.57 (br m, 5H), 4.67 (s, 2H), 3.81-3.45 (m, 10H), 3.14 (t, 2H, J = 7.8 Hz), 1.79-1.77 (m, 2H), 1.53-1.48 (m, 5H) 1.29 (br s, 20H), 0.88 (t, 3H, J = 7.0 Hz) |
| 114 | 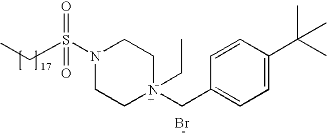 | 578(−Br) | 7.51 (d, 2H, J = 8.3 Hz), 7.44 (d, 2H, J = 8.3 Hz), 5.12 (s, 2H), 3.95-3.53 (m, 10H), 3.24 (t, 2H, J = 7.8 Hz), 1.81-1.79 (m, 2H), 1.48-1.37 (m, 5H), 1.30(s, 9H), 1.23 (br s, 28H), 0.85 (t, 3H, J = 6.7 Hz) |
| 115 | 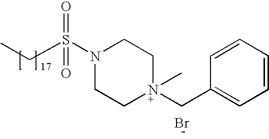 | 508(−Br) | 7.60-7.53 (m, 5H), 4.69 (s, 2H), 3.89-3.85 (m, 2H), 3.68-3.57 (m, 4H), 3.53-3.49 (m, 2H), 3.17-3.12 (m, 2H), 3.11 (s, 3H), 1.83-1.75 (m, 2H), 1.47-1.44 (m, 2H), 1.29 (br s, 28H), 0.90 (t, 3H, J = 6.7 Hz) |

### Example 116

### Preparation of 1-ethyl-1-methyl-4-palmitoyl-1,4-diazepan-1-ium iodide according to Reaction Scheme 3

### 1. Preparation of 1-(4-methyl-1,4-diazepan-1-yl)hexadecan-1-one

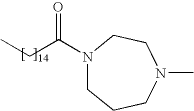

1-methylhomopiperazine (0.9□, 7.54 mmol), 1-ethyl-3-[3-dimethylaminopropyl]carbodiimide hydrochloride (1.4 g, 7.54 mmol), and 4-dimethylaminopyridine (0.2 g, 1.74 mmol) were added to a 0.1 M methylene chloride solution of palmitic acid (1.5 g, 5.80 mmol) under stirring and anhydrous conditions, and stirred at room temperature for 7 hours. The produced mixture was diluted with chloroform, washed with the saturated ammonium chloride solution three times, and then with the saturated brine solution. Then, the organic layer was dried over magnesium sulfate, and distilled off under reduced pressure. The resulting primary compound was purified by a silica gel column chromatography (eluent: 5% methanol/chloroform) to obtain the target compound in 92.4% yield (1.89 g).

¹H-NMR (300 MHz, DMSO) δ 3.48-3.41 (m, 4H), 2.45-2.40 (m, 4H), 2.28-2.22 (m, 5H), 1.81-1.71 (m, 2H), 1.47 (br s, 2H), 1.24 (br s, 24H), 0.85 (t, 3H, J=6.3 Hz)

### 2. Preparation of 1-ethyl-1-methyl-4-palmitoyl-1,4-diazepan-1-ium iodide

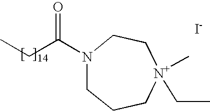

Iodoethane (0.14 g, 1.7 mmol) was added to a 0.1 M acetonitrile solution of the compound (300 mg, 0.85 mmol) obtained in the step 1 under stirring, and heated for 2 hours. The produced mixture was sufficiently cooled to 0° C., and then an ethyl acetate solution (6.0□) was added thereto under stirring for 2 hours. The produced mixture was filtered with eluding ethyl acetate solvent to obtain the target compound in 70.6% yield (304 mg).

¹H-NMR (300 MHz, DMSO) δ 3.77-3.42 (m, 10H), 3.01 (s, 3H), 2.32 (t, 2H, J=7.4 Hz), 2.12-2.09 (m, 2H), 1.49 (br s, 2H), 1.24 (br s, 27H), 0.85 (t, 3H, J=6.4 Hz)

### Examples 117 to 135

The compounds of Examples 117 to 135 were prepared in a similar manner to the preparation process that is described in Example 116.

The physical properties of the compounds are shown in Table 3.

TABLE 3

| | | MS data | |
|---|---|---|---|
| | | (M⁺H), | |
| Example | Chemical structure | (g/mol) | ¹H NMR spectrum data |
| 117 | 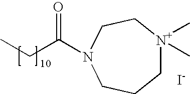 | 311(−I) | 3.73 (m, 2H), 3.53-3.38 (m, 2H), 3.32-3.29 (m, 6H), 3.09-3.08 (m, 4H), 2.29 (t, 2H, J = 7.6 Hz), 2.10 (m, 2H), 1.48 (m, 2H), 1.22 (br s, 16H), 0.83 (t, 3H, J = 6.9 Hz) |
| 118 | 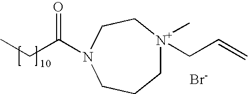 | 337(−Br) | 6.08-6.02 (m, 1H), 5.65-5.62 (m, 2H), 4.09-3.98 (m, 2H), 3.81-3.76 (m, 2H), 3.55-3.52 (m, 2H), 3.45-3.35 (br m, 3H), 3.02-2.96 (m, 4H), 2.50 (t, 2H, J = 2.0 Hz), 2.33-2.08 (m, 2H), 1.49 (br s, 2H), 1.24 (br s, 16H), 0.86-0.83 (m, 3H) |
| 119 | 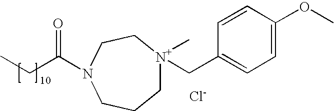 | 418(−Cl) | 7.50-7.45 (m, 2H), 7.07-7.04 (m, 2H), 4.60-4.57 (m, 2H), 3.93 (s, 3H), 3.85-3.07 (m, 2H), 3.54-3.40 (m, 2H), 3.34 (s, 3H), 3.16-2.94 (m, 4H), 2.31 (t, 3H, J = 7.4 Hz), 2.20 (br s, 2H), 1.48-1.46 (m, 2H), 1.25 (br s, 16H), 0.86 (t, 3H, J = 6.9 Hz) |
| 120 | 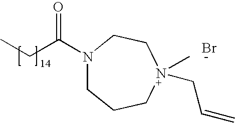 | | 6.07-6.02 (m, 1H), 5.68-5.62 (m, 2H), 4.10-4.05 (m, 2H), 3.82-3.78 (m, 2H), 3.53-3.42 (m, 6H), 3.03 (br s, 3H), 2.32 (t, 2H, J = 7.4 Hz), 2.16 (br, 2H) 1.49 (br s, 2H), 1.24 (br s, 24H), 0.85 (t, 3H, J = 6.5 Hz) |
| 121 | 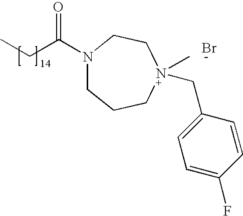 | | 7.63-7.59 (m, 2H), 7.40-7.34 (m, 2H), 4.63 (s, 2H), 3.94-3.71 (m, 2H), 3.56-3.55 (m, 2H), 3.45-3.42 (m, 4H), 2.97 (s, 3H), 2.31 (t, 2H, J = 7.4 Hz), 2.21 (br s, 2H), 1.49 (br s, 2H), 1.24 (br s, 24H), 0.85 (t, 3H, J = 6.3 Hz) |
| 122 | 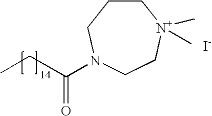 | | 3.60-3.73 (m, 2H), 3.58-3.40 (m, 6H), 3.12 (s, 6H), 2.31 (t, 2H, J = 7.5 Hz), 2.26-2.03 (m, 2H), 1.57-1.44 (m, 2H), 1.35-1.17 (m, 24H), 0.85 (t, 3H, J = 6.6 Hz) |
| 123 | 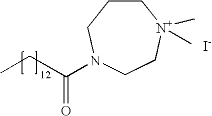 | | 3.80-3.71 (m, 2H), 3.58-3.40 (m, 6H), 3.11 (s, 6H), 2.31 (t, 2H, J = 7.5 Hz), 2.20-2.05 (m, 2H), 1.55-1.46 (m, 2H), 1.34-1.22 (m, 20H), 0.85 (t, 3H, J = 6.6 Hz) |
| 124 | 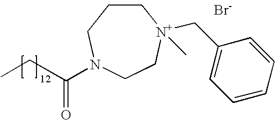 | | 7.54 (s, 5H), 4.65 (s, 2H), 4.00-3.85 (m, 1H), 3.76-3.60 (m, 1H), 3.59-3.53 (m, 2H), 3.46-3.39 (m, 4H), 2.97 (s, 3H), 2.31 (t, 2H, J = 7.2 Hz), 2.28-2.10 (m, 2H), 1.58-1.41 (m, 2H), 1.32-1.22 (m, 20H), 0.85 (t, 3H, J = 6.6 Hz) |
| 125 | 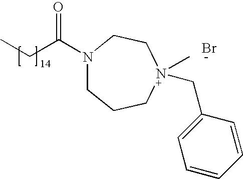 | | 7.57-7.53 (m, 5H), 4.64 (s, 2H), 3.59-3.41 (m, 6H), 2.32 (t, 2H, J = 7.4 Hz), 2.21 (br s, 2H), 1.49 (br s, 2H), 1.24 (br s, 24H), 0.85 (t, 3H, J = 6.6 Hz) |
| 126 | 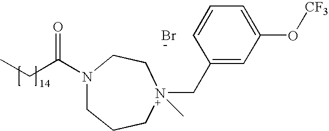 | | 7.71-7.66 (m, 2H), 7.49-7.44 (m, 2H), 7.31 (d, 1H, J = 8.6 Hz), 5.42 (br m, 2H), 4.26-3.69 (m, 8H), 3.33 (s, 3H), 2.37-2.18 (m, 4H), 1.56 (br s, 2H), 1.23 (br s, 24H), 0.85 (t, 3H, J = 6.7 Hz) |
| 127 | 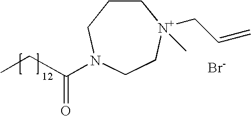 | | 6.11-6.00 (m, 1H), 5.67-5.62 (m, 2H), 4.08-4.03 (m, 2H), 3.85-3.73 (m, 2H), 3.55-3.39 (m, 6H), 3.02 (s, 3H), 2.31 (t, 2H, J = 7.5 Hz), 2.17-2.06 (m, 2H), 1.52-1.45 (m, 2H), 1.30-1.18 (m, 20H), 0.85 (t, 3H, J = 7.0 Hz) |
| 128 | 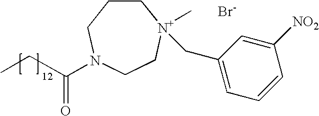 | | 8.47 (d, 1H, J = 3.7 Hz), 8.41-8.37 (m, 1H), 8.03 (t, 1H, J = 7.5 Hz), 7.85-7.80 (m, 1H), 3.61-3.42 (m, 2H), 4.02-3.84 (m, 1H), 3.74-3.62 (m, 1H), 3.61-3.42 (m, 6H), 3.04 (s, 3H), 2.37-2.31 (m, 2H), 2.25-2.18 (m, 2H), 1.54-1.45 (m, 2H), 1.33-1.19 (m, 2H), 0.85 (t, 3H, J = 6.5 Hz) |
| 129 | 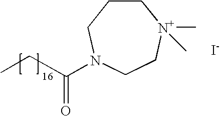 | | 3.80-3.72 (m, 2H), 3.55-3.42 (m, 6H), 3.12 (s, 6H), 2.31 (t, 2H, J = 7.5), 2.18-2.05 (m, 2H), 1.52-1.46 (m, 2H), 1.31-1.19 (m, 28H), 0.85 (t, 3H, J = 7.5 Hz) |
| 130 | 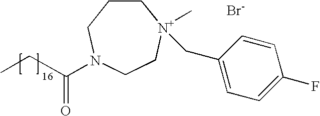 | | 7.57-7.49 (m, 5H), 4.68-4.63 (m, 2H), 3.99-3.68 (m, 2H), 3.64-3.38 (m, 6H), 2.98 (s, 3H), 2.34-2.29 (m, 2H), 2.28-2.18 (m, 2H), 1.53-1.43 (m, 2H), 1.31-1.18 (m, 28H), 0.85 (t, 3H, J = 6.5 Hz) |
| 131 | 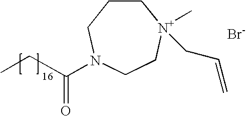 | | 6.08-6.02 (m, 1H), 5.68-5.61 (m, 2H), 4.10-4.01 (m, 2H), 3.87-3.71 (m, 2H), 3.55-3.40 (m, 6H), 3.02 (s, 3H), 2.31 (t, 2H, J = 7.5 Hz), 2.19-2.11 (m, 2H), 1.53-1.45 (m, 2H), 1.31-1.20 (m, 28H), 0.85 (t, 3H, J = 6.5 Hz) |
| 132 | 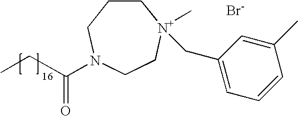 | | 7.41-7.34 (m, 4H), 4.65-4.57 (m, 4H), 4.02-3.96 (m, 1H), 3.90-3.68 (m, 1H), 3.63-3.40 (m, 6H), 2.97 (s, 3H), 2.36 (s, 3H), 2.31 (t, 2H, J = 7.5 Hz), 2.26-2.17 (m, 2H), 1.54-1.43 (m, 2H), 1.21-1.18 (m, 28H), 0.84 (t, 3H, J = 7.5 Hz) |
| 133 | 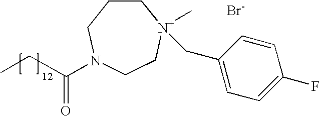 | | 7.66-7.61 (m, 2H), 7.38-7.34 9m, 2H), 4.71-4.66 9m, 2H), 4.01-3.88 (m, 1H), 3.76-3.68 9m, 1H), 3.61-3.38 (m, 6H), 2.98 (s, 3H), 2.34-2.29 (m, 2H), 2.23-2.13 (m, 2H), 1.52-1.45 (m, 2H), 1.30-1.19 (m, 20H), 0.85 (t, 3H, J = 6.5 Hz) |
| 134 | 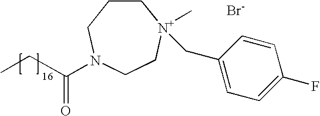 | | 7.63-7.58 (m, 2H), 7.39-7.34 (m, 2H), 4.66-4.58 (m, 2H), 3.98-3.86 (m, 1H), 3.85-3.66 (m, 1H), 3.59-3.38 (m, 6H), 2.95 (s, 3H), 2.31 (t, 2H, J = 7.5 Hz), 2.23-2.12 (m, 2H), 1.52-1.42 (m, 2H), 1.30-1.17 (m, 28H), 0.85 (t, 3H, J = 7.0 Hz) |
| 135 | 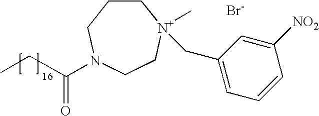 | | 8.51-8.45 (m, 1H), 8.41-8.35 (m, 1H), 8.11-8.04 (m, 1H), 7.86-7.79 (m, 1H), 5.07-4.85 (m, 2H), 4.03-3.87 (m, 1H), 3.73-3.67 (m, 1H), 3.65-3.42 9m, 6H), 3.07 (s, 3H), 2.36-2.17 (m, 4H), 1.53-1.45 (m, 2H), 1.38-1.16 (m, 28H), 0.84 (t, 3H, J = 6.6 Hz) |

### Experimental Example 1

### Pharmacological Activity Test

In order to test the efficacy of the compounds according to the present invention, experiments were performed as follows.

1-1. Test for Inhibition of Tumor Cell Growth

Human tumor cell lines, PC-3 (prostate cancer, ATCC, USA), MBA-MB-231 (breast cancer, ATCC, USA), ACHN (kidney cancer, ATCC, USA), and NUGC-3 (gastric cancer, ATCC, USA) were cultured in RPMI 1640 media containing 10% fetal bovine serum (FBS).

In order to measure anticancer activity, a suitable concentration of cells in RPMI 1640 media containing 5% fetal bovine serum (about 5×10⁴ cells/□) was aliquotted in 96-well plates, and cultured in 5% CO₂ at 37° C. On day one after aliquoting the cells, before the cells were treated with the compounds, in order to determine their concentration, 50□ of 50% trichloroacetic acid was added to each well of time zero (T₀) plate, and cells were fixed to determine zero point. The cells treated with the compounds were fixed on each well having 50□ of 50% trichloroacetic acid after 48 hours. The final concentrations of the test compounds were 0.01, 0.03, 0.1, 0.3, and 1□/□. The fixed plate was washed with water and dried, and then 100□ of 0.4% sulphorhodamine B (SRB) dissolved in 0.1% acetic acid was added to the each well to stain the cells. The plate was allowed to stand for 30 minutes, and then washed with 0.1% acetic acid. Then, the plate was dried at room temperature, and treated with 10 mM tris base (pH 10.5) to dissolve staining reagent. An absorbance measured at 540 nm was calculated as a percentage of the control group, and then the concentration of the compound that inhibited tumor cell growth (GI₅₀(□/□)) by 50% was determined. The results are shown in Table 4.

TABLE 4

| | GI₅₀ (μg/ml) | | | |
|---|---|---|---|---|
| | PC-3 | | | |
| | (prostate | MBA-MB-231 | ACHN | NUGC-3 |
| Example | ancer) | (breast cancer) | (kidney cancer) | (gastric cancer) |
| 1 | 0.99 | 3.51 | 2.39 | 1.15 |
| 2 | >10 | >10 | >10 | >10 |
| 3 | >10 | >10 | >10 | >10 |
| 4 | >10 | >10 | >10 | >10 |
| 5 | >10 | >10 | >10 | >10 |
| 6 | >10 | >10 | >10 | >10 |
| 7 | >10 | >10 | >10 | >10 |
| 8 | 0.80 | >10 | >10 | >10 |
| 9 | 1.81 | >10 | >10 | >10 |
| 10 | >10 | >10 | >10 | >10 |
| 11 | 3.07 | >10 | >10 | >10 |
| 12 | >10 | >10 | >10 | >10 |
| 13 | 0.30 | 1.29 | >10 | 0.50 |
| 14 | 0.57 | 2.44 | 2.37 | 0.54 |
| 15 | 1.42 | >10 | >10 | 2.07 |
| 16 | 0.41 | 2.49 | 4.26 | 0.76 |
| 17 | 0.46 | 1.28 | 1.14 | 0.47 |
| 18 | 0.49 | 0.92 | 1.31 | 1.16 |
| 19 | 0.55 | 1.70 | 1.69 | 0.63 |
| 20 | 0.67 | 1.52 | 1.58 | 0.72 |
| 21 | 0.55 | 1.16 | 0.92 | 0.46 |
| 22 | 0.37 | 0.68 | 0.50 | 0.27 |
| 23 | 0.80 | 2.05 | 1.89 | 0.58 |
| 24 | 0.45 | 0.76 | 0.78 | 0.40 |
| 25 | 0.40 | 0.82 | 2.84 | 1.05 |
| 26 | 0.54 | 1.13 | 1.79 | 0.88 |
| 27 | 0.41 | 1.43 | 1.36 | 0.67 |
| 28 | 3.64 | >10 | >10 | 5.21 |
| 29 | 0.84 | 2.51 | 2.43 | 1.37 |
| 30 | 0.34 | 1.71 | 1.23 | 0.61 |
| 31 | 0.31 | 1.63 | 1.39 | 0.42 |
| 32 | 0.59 | 1.66 | 1.89 | 1.10 |
| 33 | 4.29 | >10 | >10 | 3.45 |
| 34 | 0.85 | 2.01 | 1.90 | 1.22 |
| 35 | 1.24 | 1.98 | 4.74 | 1.90 |
| 36 | 0.96 | 3.16 | 2.37 | 1.53 |
| 37 | >10 | >10 | >10 | >10 |
| 38 | 1.47 | 2.53 | 3.38 | 1.54 |
| 39 | 4.79 | >10 | >10 | >10 |
| 40 | 4.79 | >10 | >10 | >10 |
| 41 | 3.36 | >10 | >10 | >10 |
| 42 | 3.77 | >10 | >10 | >10 |
| 43 | 1.32 | >10 | >10 | >10 |
| 44 | 0.41 | >10 | >10 | >10 |
| 45 | 2.02 | >10 | >10 | >10 |
| 46 | 0.51 | 1.45 | 1.98 | 1.00 |
| 47 | 4.57 | >10 | 5.13 | 3.64 |
| 48 | 0.68 | 1.49 | 1.43 | 1.19 |
| 49 | 3.14 | 5.38 | 3.08 | 3.63 |
| 50 | 0.95 | 2.30 | 1.94 | 2.49 |
| 51 | 0.52 | 1.42 | 1.85 | 1.05 |
| 52 | 0.51 | 1.60 | 2.08 | 0.88 |
| 53 | 0.57 | 3.95 | 4.84 | 3.52 |
| 54 | 0.53 | 4.71 | 3.78 | 1.61 |
| 55 | 0.41 | 4.37 | 3.84 | 0.90 |
| 56 | 0.52 | 1.13 | 1.93 | 1.22 |
| 57 | 0.99 | 2.54 | 2.07 | 1.13 |
| 58 | 0.63 | 1.52 | 0.93 | 0.46 |
| 59 | 0.84 | 2.05 | 1.07 | 0.66 |
| 60 | >10 | >10 | >10 | >10 |
| 61 | >10 | >10 | >10 | >10 |
| 62 | >10 | >10 | >10 | >10 |
| 63 | >10 | >10 | >10 | >10 |
| 64 | 0.67 | 2.07 | 1.43 | 0.36 |
| 65 | 0.42 | 0.82 | 1.16 | 0.37 |
| 66 | 0.56 | 1.47 | 1.24 | 1.06 |
| 67 | 0.87 | 1.05 | 0.51 | 0.60 |
| 68 | 0.71 | 1.59 | 1.19 | 1.05 |
| 69 | 0.68 | 1.16 | 0.59 | 0.59 |
| 70 | 0.49 | 0.87 | 1.11 | 0.53 |
| 71 | 0.65 | 1.14 | 1.42 | 0.82 |
| 72 | 0.73 | 1.37 | 0.71 | 0.57 |
| 73 | 0.80 | 1.75 | 1.87 | 0.85 |
| 74 | 1.00 | 1.72 | 1.25 | 0.85 |
| 75 | 1.84 | >10 | 4.45 | 3.29 |
| 76 | 0.54 | 4.02 | 3.50 | 0.99 |
| 77 | 0.83 | >10 | 3.47 | 1.55 |
| 78 | 1.06 | >10 | 4.15 | 1.81 |
| 79 | 0.85 | >10 | 4.41 | 1.24 |
| 80 | 0.85 | >10 | 3.07 | 0.89 |
| 81 | 0.61 | 1.49 | 1.48 | 0.58 |
| 82 | 0.71 | 1.40 | 0.86 | 0.75 |
| 83 | 0.61 | 1.42 | 0.69 | 0.45 |
| 84 | 0.74 | 1.35 | 1.12 | 0.75 |
| 85 | 0.41 | 1.01 | 0.53 | 0.22 |
| 86 | 0.88 | 4.48 | 3.79 | 1.99 |
| 87 | 1.25 | >10 | >10 | 1.56 |
| 88 | 0.68 | 1.59 | 1.37 | 0.93 |
| 89 | 4.02 | >10 | >10 | >10 |
| 90 | 1.57 | 2.56 | 3.22 | 1.98 |
| 91 | 0.83 | >10 | 4.82 | 1.54 |
| 92 | 0.45 | 1.62 | 0.81 | 0.21 |
| 93 | 0.67 | 2.71 | 1.11 | 0.62 |
| 94 | 0.53 | 0.46 | 0.31 | 0.37 |
| 95 | 0.35 | 0.64 | 0.46 | 2.24 |
| 96 | 0.48 | 0.60 | 3.25 | 2.73 |
| 97 | 0.70 | 1.43 | 1.21 | 0.66 |
| 98 | 0.35 | 1.01 | 1.05 | 0.28 |
| 99 | >10 | >10 | >10 | >10 |
| 100 | 4.08 | >10 | >10 | 1.73 |
| 101 | 2.34 | >10 | >10 | 3.28 |
| 102 | >10 | >10 | >10 | >10 |
| 103 | 1.25 | >10 | >10 | 3.45 |
| 104 | 3.45 | >10 | >10 | >10 |
| 105 | 0.65 | 4.76 | >10 | 1.56 |
| 106 | 0.61 | 3.56 | 4.13 | 1.60 |
| 107 | 1.05 | >10 | >10 | 2.72 |
| 108 | 0.45 | 1.19 | 1.42 | 0.65 |
| 109 | 1.95 | >10 | 4.10 | 1.51 |
| 110 | 0.80 | >10 | 3.94 | 0.98 |
| 111 | 0.00 | 0.00 | 0.00 | 0.00 |
| 112 | 0.43 | 1.75 | 1.69 | 1.75 |
| 113 | 0.41 | 1.23 | 1.05 | 1.22 |
| 114 | 2.01 | 2.24 | 2.32 | 3.16 |
| 115 | 0.48 | 0.91 | 2.01 | 1.07 |
| 116 | 0.36 | 0.59 | 0.29 | 0.25 |
| 117 | 0.50 | 2.68 | 1.07 | 0.60 |
| 118 | 0.78 | 3.84 | 2.18 | 0.87 |
| 119 | 0.57 | 2.48 | 1.48 | 0.36 |
| 120 | 0.75 | 1.44 | 0.74 | 0.20 |
| 121 | 0.46 | 0.83 | 1.09 | 0.63 |
| 122 | 0.40 | 0.94 | 0.40 | 0.21 |
| 123 | 0.36 | 0.98 | 0.49 | 0.08 |
| 124 | 0.45 | 1.16 | 1.00 | 0.31 |
| 125 | 0.35 | 0.86 | 0.80 | 0.53 |
| 126 | 0.63 | 1.34 | 1.93 | 0.87 |
| 127 | 0.18 | 0.45 | 0.44 | 0.26 |
| 128 | 0.18 | 0.71 | 0.82 | 0.46 |
| 129 | 0.24 | 0.67 | 0.57 | 0.26 |
| 130 | 0.20 | 0.73 | 0.81 | 0.66 |
| 131 | 0.20 | 0.66 | 0.66 | 0.37 |
| 132 | 0.35 | 1.27 | 1.06 | 0.66 |
| 133 | 0.45 | 1.48 | 1.10 | 0.94 |
| 134 | 0.57 | 1.11 | 1.42 | 0.97 |
| 135 | >10 | >10 | >10 | >10 |

1-2. Test for Tumor Growth Inhibition in Animal Model

Female S.P.F BALB/c nude mice (7-week-old) were grafted with 3×10⁷ cells/□ of human prostate cancer cell line PC-3, and then intraperitoneally administered with the compound prepared in Example 55 at a daily dosage of 30 mg/kg 20 times. A positive control group was intraperitoneally administered with adriamycin at a daily dosage of 2 mg/kg 10 times once every two days.

In order to test toxicity, animals were observed for changes in body weight, deaths, and size and weight of the tumor during the administration period.

The changes in their body weight are shown in FIG. 1, the changes in tumor size are shown in FIG. 2, and the tumor weights measured on the final day (day 21) are shown in FIG. 3.

As shown in FIG. 1, specific symptoms were not observed in mice, which had been intraperitoneally administered with the compound according to the present invention (Example 55), for the experimental period. Further, from the result of measuring the changes in mouse weight on the final day (day 21), no weight changes were observed in the group treated with the compound of the present invention, as compared to the vehicle-control group. In the group treated with the positive control substance (adriamycin/2 mg/kg/Q2D: 10 times), four deaths and 13.7% (p<0.001) weight loss were observed.

As shown in FIG. 2, from the result on the final day (day 21), a statistically significant effect of inhibiting tumor growth was observed (84.0%, p<0.001), in the group treated with the compound of the present invention (30 mg/kg), as compared to the vehicle-control group. In the group treated with the positive control substance (adriamycin/2 mg/kg/Q2D: 10 times), a statistically significant effect of inhibiting tumor growth was observed (74.4%, p<0.01).

The mice were sacrificed on the final day (day 21), their tumors were removed, and then the tumor weight was measured. Consequently, as shown in FIG. 3, in the group treated with the compound of the present invention (30 mg/kg), a statistically significant effect was observed in the reduction of tumor weight (79.5%, p<0.001); as compared to the vehicle-control group. In the group treated with the positive control substance (adriamycin/2 mg/kg/Q2D: 10 times), a statistically significant effect was observed in the reduction of tumor weight (69.8%, p<0.05).

### Experimental Example 2

### Western Blot Analysis

4×10⁶ of human prostate cancer cell line, PC-3 cells were cultured in 100 mm diameter-culture dishes with RPMI media containing 5% FBS for one day, and then treated with the compound prepared in Example 55 at concentrations of 0, 2, and 5 uM for 24 hours, respectively. Then, the cells were carefully washed with 10□ of PBS twice, and 1 ml of PBS containing protease inhibitor cocktail (Roche, complete™-mini) (1 tablet/50□ PBS) was added to the each dish. The cells were collected and sonicated. The sonicated cells were centrifuged using a microcentrifuge at 12000 rpm for 20 minutes, and the supernatant was collected. Then, the amount of protein was determined using a Bradford dye reagent (Bio-Rad), and after running 20□ of the protein on an SDS-PAGE gel, the protein band was transferred to a nitrocellulose membrane (Bio-Rad). Subsequently, the amounts of each protein were analyzed using primary antibodies and secondary antibodies-HRP (horseradish peroxidase) that are specific to each protein to be tested (Amersham or Bio-Rad), and an ECL chemiluminescence reagent (Amersham).

The results are shown in FIG. 4.

Human prostate cancer cell line, PC-3 cells were treated with the compound according to the present invention (Example 55), and then western blot analysis was performed to measure the amount of the protein. Consequently, as shown in FIG. 4, the amount of c-abl in response to DNA damage increased, and the amount of p53 and phosphorylated p53 sharply increased. Further, the amount of RhoB increased, which has been reported to be involved in apoptosis, and apoptosis was generated by RhoB induction (FIG. 4A). Further, the amount of Bcl2 involved in the cell survival was down-regulated by dysregulated signals via the mitochondria pathway (FIG. 4B).

### Experimental Example 3

### Flow Cytometric Analysis

5×10⁵ of human prostate cancer cell line, PC-3 cells were cultured in 60 mm diameter-culture dishes with RPMI 1640 media containing 5% FBS in an incubator supplied with 5% CO₂ at 37° C. for one day, and then treated with 5 uM NAC (N-acetylcysteine, Sigma) for 3 hours. Then, the cells were treated with the compound prepared in Example 55 at concentrations of 5 uM, cultured for 24 hours. Subsequently, the cells were treated with 0.1% trypsin and detached. The detached cells were transferred to a 15□ conical tube, and centrifuged at 200×g for 5 minutes to precipitate the cells. Then, the supernatant was removed. 0.5□ of PBS solution containing 0.1 mg/□ RNase A was added thereto, so as to resuspend the cells. The cells were treated with a PI (propidium iodide) DNA staining solution in a concentration of 50□/□ to stain the DNA in the cells for 30 or more minutes. The PI-stained cells were e xcited using a flow cytometer (Becton Dickinson) at 488 nm by a laser beam, and an emission wavelength of 588 nm was represented by a histogram, and then quantitatively analyzed to determine the amount of DNA in the cells.

The results are shown in FIG. 5.

As shown in FIG. 5, when the human prostate cancer cell line, PC-3 cells were treated with an antioxidant NAC (N-acetylcysteine) that functions to remove reactive oxygen species (ROS), the degree of apoptosis was greatly decreased, as compared to the prostate cancer cell line PC-3 cells treated with only the compound according to the present invention (Example 55).

Accordingly, it can be assumed that the compound according to the present invention induces apoptosis due to the DNA damage by the reactive oxygen species.

Formulation Examples for the composition of the present invention are described as follows.

### Formulation Example 1

### Preparation of Injectable Formulation

An injectable formulation containing 10 mg of the active ingredient was prepared as the following method.

1 g of the compound of Formula 1, 0.6 g of sodium chloride, and 0.1 g of ascorbic acid were dissolved in distilled water and made up to a volume of 100 ml. The solution was put into a bottle, and heated at 20° C. for 30 minutes for sterilization.

The compositions of the injectable formulation are as follows.

Compound of Formula 1 1 g

Sodium chloride 0.6 g

Ascorbic acid 0.1 g

Distilled water Predetermined amount

### Formulation Example 2

### Preparation of Syrup Formulation

A syrup formulation containing the compound of Formula 1 (2%, weight/volume) as an active ingredient was prepared as the following method.

The compound of Formula 1, saccharin, and sugar were dissolved in 80 g of warm water. The solution was cooled, and mixed with a solution consisting of glycerin, saccharin, flavor, ethanol, sorbic acid, and distilled water. The mixture was made up to a volume of 100 ml with water.

The compositions of the syrup are as follows.

Compound of Formula 12 g

Saccharin 0.8 g

Sugar 25.4 g

Glycerin 8.0 g

Flavor 0.04 g

Ethanol 4.0 g

Sorbic acid 0.4 g

Distilled water Predetermined amount

### Formulation Example 3

### Preparation of Tablet

A tablet containing 15 mg of the active ingredient was prepared as the following method.

250 g of the compound of Formula 1 was mixed with 175.9 g of lactose, 180 g of potato starch, and 32 g of colloidal silicate. A 10% gelatin solution was added thereto, and then pulverized, passed through a 14 mesh sieve. The resultant was dried, and 160 g of potato starch, 50 g of talc, and 5 g of magnesium stearate were added thereto. The mixture was compressed into a tablet.

The compositions of the tablet are as follows.

Compound of Formula 1 250 g

Lactose 175.9 g

Potato starch 180 g

Colloidal silicate 32 g

10% Gelatin solution

Potato starch 160 g

Talc 50 g

Magnesium stearate 5 g

### INDUSTRIAL APPLICABILITY

The compounds according to the present invention induce DNA damage due to reactive oxygen species to activate c-abl and p53, induce RhoB to generate apoptosis, and induce cell death by down-regulating Bcl2 involved in cell survival, which is generated by dysregulated signals via the mitochondria pathway, thereby inhibiting tumor cell growth and inducing apoptosis. Accordingly, the composition according to the present invention can be used to treat cancer.

1. Heterocyclic compounds containing nitrogen atoms represented by Formula 1 or pharmaceutically acceptable salts thereof. 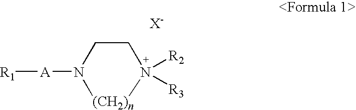
For the Formula 1,
R₁ is straight or branched chain C₁˜C₃₀ alkyl, or C₂˜C₃₀ alkenyl,
R₂ is straight or branched chain C₁˜C₆ alkyl,
R₃ is straight or branched chain C₁˜C₆ alkyl; C₁˜C₆ alkenyl; allyl; or benzyl substituted or unsubstituted with one group selected from the group consisting of straight or branched chain C₁˜C₆ alkyl, C₁˜C₆ alkoxy, OCF₃, nitro, and halogen atom,
A is C(═O) or S(═O)₂,
X is a halogen atom, and
n is an integer of 2 and 3.

2. The heterocyclic compounds containing nitrogen atoms or the pharmaceutically acceptable salts thereof according to claim 1, wherein the compounds of Formula 1 are selected from the group consisting of
1) 4-docosanoyl-1,1-dimethylpiperazin-1-ium iodide,
2) 1,1-dimethyl-4-octanoyl-piperazin-1-ium iodide,
3) 1-benzyl-1-methyl-4-octanoyl-piperazin-1-ium bromide,
4) 1-allyl-1-methyl-4-octanoyl-piperazin-1-ium bromide,
5) 1-(4-methoxy-benzyl)-1-methyl-4-octanoyl-piperazin-1-ium chloride,
6) 1-ethyl-1-methyl-4-octanoyl-piperazin-1-ium iodide,
7) 1-benzyl-1-ethyl-4-octanoyl-piperazin-1-ium bromide,
8) 1-benzyl-4-decanoyl-1-methyl-piperazin-1-ium bromide,
9) 1-allyl-4-decanoyl-1-methyl-piperazin-1-ium bromide,
10) 1-benzyl-4-decanoyl-1-ethyl-piperazin-1-ium bromide,
11) 1-allyl-4-decanoyl-1-ethylpiperazin-1-ium bromide,
12) 4-decanoyl-1-ethyl-1-methylpiperazin-1-ium iodide,
13) 1-allyl-1-methyl-4-tetradecanoyl-piperazin-1-ium bromide,
14) 1-ethyl-1-methyl-4-tetradecanoyl-piperazin-1-ium iodide,
15) 1-benzyl-1-ethyl-4-tetradecanoyl-piperazin-1-ium bromide,
16) 1-allyl-1-ethyl-4-tetradecanoyl-piperazin-1-ium bromide,
17) 1-allyl-4-hexadecanoyl-1-methyl-piperazin-1-ium bromide,
18) 4-hexadecanoyl-1-(4-methoxy-benzyl)-1-methyl-piperazin-1-ium chloride,
19) 4-hexadecanoyl-1-methyl-1-pent-4-enyl-piperazin-1-ium bromide,
20) 1-but-3-enyl-4-hexadecanoyl-1-methyl-piperazin-1-ium bromide,
21) 1-benzyl-1-ethyl-4-hexadecanoyl-piperazin-1-ium bromide,
22) 1-allyl-1-ethyl-4-hexadecanoyl-piperazin-1-ium bromide,
23) 1-ethyl-4-hexadecanoyl-1-methyl-piperazin-1-ium iodide,
24) 1-ethyl-4-hexadecanoyl-1-pent-4-enyl-piperazin-1-ium bromide,
25) 1-benzyl-1-methyl-4-octadecanoyl-piperazin-1-ium bromide,
26) 1-allyl-1-methyl-4-octadecanoyl-piperazin-1-ium bromide,
27) 1-ethyl-1-methyl-4-octadecanoyl-piperazin-1-ium iodide,
28) 1-benzyl-1-ethyl-4-octadecanoyl-piperazin-1-ium bromide,
29) 1-allyl-1-ethyl-4-octadecanoyl-piperazin-1-ium bromide,
30) 1-ethyl-4-icosanoyl-1-methylpiperazin-1-ium iodide,
31) 4-icosanoyl-1,1-dimethylpiperazin-1-ium iodide,
32) 1-benzyl-4-icosanoyl-1-thethylpiperazin-1-ium bromide,
33) 1-allyl-4-icosanoyl-1-methylpiperazin-1-him bromide,
34) 4-docosanoyl-1-ethyl-1-methylpiperazin-1-ium iodide,
35) 1-benzyl-4-docosanoyl-1-methylpiperazin-1-ium bromide,
36) 1-allyl-4-docosanoyl-1-ticiethylpiperazin-1-ium bromide,
37) 1-benzyl-4-docosanoyl-1-ethylpiperazin-1-ium bromide,
38) 1-allyl-4-docosanoyl-1-ethylpiperazin-1-ium bromide,
39) 1,1-dimethyl-4-tetracosanoylpiperazin-1-ium iodide,
40) 1-benzyl-1-methyl-4-tetracosanoylpiperazin-1-ium bromide,
41) 1-allyl-1-methyl-4-tetracosanoylpiperazin-1-ium bromide,
42) 1-ethyl-1-methyl-4-tetracosanoylpiperazin-1-ium iodide,
43) 1-allyl-1-methyl-4-undec-10-enoyl-piperazin-1-ium bromide,
44) 1-benzyl-1-methyl-4-undec-10-enoyl-piperazin-1-ium bromide,
45) 1,1-dimethyl-4-undec-10-enoyl-piperazin-1-ium iodide,
46) 1-benzyl-1-methyl-4-palmitoylpiperazin-1-ium bromide,
47) 1-ethyl-1-(3-nitrobenzyl)-4-palmitoylpiperazin-1-ium bromide,
48) 1-ethyl-1-(4-fluorobenzyl)-4-stearoylpiperazin-1-ium bromide;
49) 1-ethyl-1-(3-nitrobenzyl)-4-stearoylpiperazin-1-ium bromide,
50) 1-(4-bromobenzyl)-1-ethyl-4-stearoylpiperazin-1-ium bromide,
51) 1-ethyl-1-(3-fluorobenzyl)-4-tetradecanoylpiperazin-1-ium bromide,
52) 1-ethyl-1-(3-methylbenzyl)-4-tetradec anoylpiperazin-1-ium bromide,
53) 4-dodecanoyl-1-ethyl-1-(3-nitrobenzyl)piperazin-1-ium bromide,
54) 4-dodecanoyl-1-ethyl-1-(3-fluorobenzyl)piperazin-1-ium bromide,
55) 4-dodecanoyl-1-ethyl-1-(3-methylbenzyl)piperazin-1-ium bromide,
56) 1-ethyl-1-(3-nitrobenzyl)-4-tetradecanoylpiperazin-1-ium bromide,
57) 1-ethyl-4-octadecyl-1-(3-(trifluoromethoxy)benzyl)piperazin-1-ium bromide,
58) 1-ethyl-1-(3-methylbenzyl)-4-stearoylpiperazin-1-ium bromide,
59) 1-ethyl-1-methyl-4-(octadecane-1-sulfonyl)piperazin-1-ium iodide,
60) 1-allyl-1-ethyl-4-(octane-1-sulfonyl)piperazin-1-ium bromide,
61) 1-allyl-1-methyl-4-(octane-1-sulfonyl)piperazin-1-ium bromide,
62) 1-benzyl-1-methyl-4-(octane-1-sulfonyl)piperazin-1-ium bromide,
63) 1-(4-fluorobenzyl)-1-methyl-4-(octane-1-sulfonyl)piperazin-1-ium bromide,
64) 1-benzyl-1-ethyl-4-(undecane-1-sulfonyl)piperazin-1-ium bromide,
65) 1-benzyl-1-methyl-4-(undecane-1-sulfonyl)piperazin-1-ium bromide,
66) 1-(4-fluorobenzyl)-1-methyl-4-(undecane-1-sulfonyl)piperazin-1-ium bromide,
67) 1-allyl-1-ethyl-4-(tetradecane-1-sulfonyl)piperazin-1-ium bromide,
68) 1-ethyl-1-(4-fluorobenzyl)-4-(tetradecane-1-sulfonyl)piperazin-1-ium bromide,
69) 1-allyl-1-methyl-4-(tetralecane-1-sulfonyl)piperazin-1-ium bromide,
70) 1-benzyl-1-methyl-4-(tetradecane-1-sulfonyl)piperazin-1-ium bromide,
71) 1-(4-fluorobenzyl)-1-methyl-4-(tetradecane-1-sulfonyl)piperazin-1-ium bromide,
72) 1-allyl-1-ethyl-4-(octadecane-1-sulfonyl)piperazin-1-ium bromide,
73) 1-benzyl-1-ethyl-4-(octadecane-1-sulfonyl)piperazin-1-ium bromide,
74) 1-allyl-1-methyl-4-(octadecane-1-sulfonyl)piperazin-1-ium bromide,
75) 4-(decane-1-sulfonyl)-1,1-dimethyl-piperazin-1-ium iodide,
76) 1-benzyl-4-(decane-1-sulfonyl)-1-methyl-piperazin-1-ium bromide,
77) 1-allyl-4-(decane-1-sulfonyl)-1-methyl-piperazin-1-him bromide,
78) 4-(decane-1-sulfonyl)-1-ethyl-1-methyl-piperazin-1-ium iodide,
79) 1-benzyl-4-(decane-1-sulfonyl)-1-ethyl-piperazin-1-ium bromide,
80) 1-allyl-4-(decane-1-sulfonyl)-1-ethyl-piperazin-1-ium bromide,
81) 1-benzyl-4-(dodecane-1-sulfonyl)-1-methyl-piperazin-1-ium bromide,
82) 1-all yl-4-(dodecane-1-sulfonyl)-1-methyl-piperazin-1-ium bromide,
83) 4-(dodecane-1-sulfonyl)-1-ethyl-1-methyl-piperazin-1-ium iodide,
84) 1-benzyl-4-(dodecane-1-sulfonyl)-1-ethyl-piperazin-1-ium bromide,
85) 1-allyl-4-(dodecane-1-sulfonyl)-1-ethyl-piperazin-1-ium bromide,
86) 4-(decane-1-sulfonyl)-1-(4-fluorobenzyl)-1-methyl-piperazin-1-ium bromide,
87) 4-(decane-1-sulfonyl)-1-ethyl-1-(4-fluorobenzyl)-piperazin-1-ium bromide,
88) 4-(dodecane-1-sulfonyl)-1-(4-fluorobenzyl)-1-methyl-piperazin-1-ium bromide,
89) 1-ethyl-1-(3-nitrobenzyl)-4-(nonylsulfonyl)piperazin-1-ium bromide,
90) 4-(dodecane-1-sulfonyl)-1-methyl-1-(4-methylbenzyl)piperazin-1-ium bromide,
91) 4-(decane-1-sulfonyl)-1-ethyl-1-(3-nitrobenzyl)piperazin-1-ium bromide,
92) 1,1-diethyl-4-(undecane-1-sulfonyl)piperazin-1-ium iodide,
93) 1-ethyl-1-methyl-4-(undecane-1-sulfonyl)piperazin-1-ium iodide,
94) 1,1-diethyl-4-(tetradecane-1-sulfonyl)piperazin-ium iodide,
95) 1-ethyl-1-methyl-4-(tetradecane-1-sulfonyl)piperazin-1-ium iodide,
96) 1-(4-fluorobenzyl)-1-methyl-4-(octadecane-1-sulfonyl)piperazin-1-ium bromide,
97) 4-(dodecane-1-sulfonyl)-1-ethyl-1-(4-fluorobenzyl)piperazin-1-ium bromide,
98) 4-(dodecane-1-sulfonyl)-1-ethyl-1-(3-nitrobenzyl)piperazin-1-ium bromide,
99) 1-ethyl-1-methyl-4-(nonane-1-sulfonyl)piperazin-1-ium iodide,
100) 1-benzyl-1-ethyl-4-(nonane-1-sulfonyl)piperazin-1-ium bromide,
101) 1-allyl-1-ethyl-4-(nonane-1-sulfonyl)piperazin-1-ium bromide,
102) 1,1-dimethyl-4-(nonane-1-sulfonyl)piperazin-1-ium iodide,
103) 1-benzyl-1-methyl-4-(nonane-1-sulfonyl)piperazin-1-ium bromide,
104) 1-(4-fluorobenzyl)-1-methyl-4-(nonane-1-sulfonyl)piperazin-1-ium bromide,
105) 4-(decane-1-sulfonyl)-1-methyl-1-(3-nitrobenzyl)piperazin-1-ium bromide,
106) 4-(decane-1-sulfonyl)-1-methyl-1-(3-methylbenzyl)piperazin-1-ium bromide,
107) 4-(decane-1-sulfonyl)-1-methyl-1-(4-methylbenzyl)piperazin-1-ium bromide,
108) 4-(dodecane-1-sulfonyl)-1-methyl-1-(3-methylbenzyl)piperazin-1-ium bromide,
109) 4-(decane-1-sulfonyl)-1-ethyl-1-(4-methylbenzyppiperazin-1-ium bromide,
110) 4-(decane-1-sulfonyl)-1-ethyl-1-(3-methylbenzyl)piperazin-1-ium bromide,
111) 1-benzyl-1-ethyl-4-(octane-1-sulfonyl)piperazin-1-ium bromide,
112) 1-allyl-1-methyl-4-(undecane-1-sulfonyppiperazin-1-ium bromide,
113) 1-benzyl-1-ethyl-4-(tetradecane-1-sulfonyl)piperazin-1-ium bromide,
114) 1-(4-t-butylbenzyl)-1-ethyl-4-(octadecane-1-sulfonyl)piperazin-1-ium bromide,
115) 1-benzyl-1-methyl-4-(octadecane-1-sulfonyl)piperazin-1-ium bromide,
116) 1-ethyl-1-methyl-4-palmitoyl-1,4-diazepan-1-ium iodide,
117) 4-dodecanoyl-1,1-dimethyl-1,4-diazepan-1-ium iodide,
118) 1-allyl-4-dodecanoyl-1-methyl-1,4-diazepan-1-ium bromide,
119) 4-dodecanoyl-1-(4-methoxybenzyl)-1-methyl-1,4-diazepan-1-ium chloride,
120) 1-allyl-1-methyl-4-palmitoyl-1,4-diazepan-1-ium bromide,
121) 1-(4-fluorobenzyl)-1-methyl-4-palmitoyl-1,4-diazepan-1-ium bromide,
122) 4-hexadecanoyl-1,1-dimethyl-1,4-diazepan-1-ium iodide,
123) 1,1-dimethyl-4-tetradecanoyl-1,4-diazepan-1-ium iodide,
124) 1-benzyl-1-methyl-4-tetradecanoyl-1,4-diazepan-1-ium bromide,
125) 1-benzyl-1-methyl-4-palmitoyl-1,4-diazepan-1-ium bromide,
126) 4-hexadecyl-1-methyl-1-(3-(trifluoromethoxy)benzyl)-1,4-diazepan-1-ium bromide,
127) 1-allyl-1-methyl-4-tetradecanoyl-1,4-diazepan-1-ium bromide,
128) 1-methyl-1-(3-nitrobenzyl)-4-tetradecanoyl-1,4-diazepan-1-ium bromide,
129) 1,1-dimethyl-4-stearoyl-1,4-diazepan-1-ium iodide,
130) 1-(4-fluorobenzyl)-1-methyl-4-stearoyl-1,4-diazepan-1-ium bromide,
131) 1-allyl-1-methyl-4-octadecyl-1,4-diazepan-1-ium bromide,
132) 1-methyl-1-(3-methylbenzyl)-4-octadecyl-1,4-diazepan-1-ium bromide,
133) 1-(4-fluorobenzyl)-1-methyl-4-tetradecanoyl-1,4-diazepan-1-ium bromide,
134) 1-(4-fluorobenzyl)-1-methyl-4-octadecyl-1,4-diazepan-1-ium bromide, and
135) 1-methyl-1-(3-nitrobenzyl)-4-octadecyl-1,4-diazepan-1-ium bromide.

3. A process for the preparation of the compounds of claim 1 represented by Reaction Scheme 1, the process comprising the steps of:
1) reacting an organic acid compound of Formula 2 with thionyl chloride in an organic solvent, and then reacting with an alkylpiperazine derivative to prepare a compound of Formula 3; and
2) reacting the compound of Formula 3 prepared in the step 1) with a halide compound to prepare a compound of Formula 1-1. 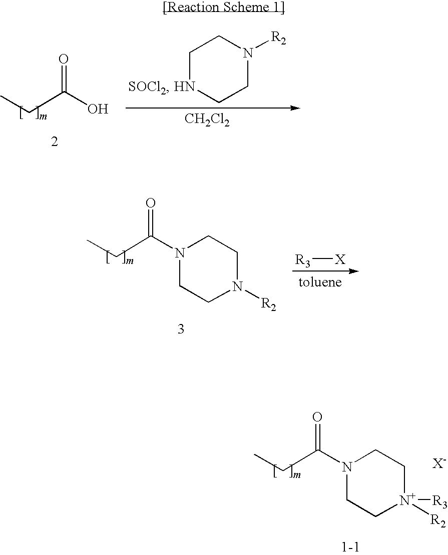
for Reaction Scheme 1,
R₂ is straight or branched chain C₁˜C₆ alkyl,
R₃ is straight or branched chain C₁˜C₆ alkyl; C₂˜C₃₀ alkenyl; allyl; or benzyl substituted or unsubstituted with one group selected from the group consisting of straight or branched chain C₁˜C₆ alkyl, C₁˜C₆ alkoxy, OCF₃, nitro, and halogen atom,
X is a halogen atom, and
m is an integer of 1 to 30.

4. A process for the preparation of the compounds of claim 1 represented by Reaction Scheme 2, the process comprising the steps of:
1) reacting a sulfonic acid compound of Formula 4 with oxalyl chloride in an organic solvent, and then reacting with an alkylpiperazine derivative to prepare a compound of Formula 5; and
2) reacting the compound of Formula 5 prepared in the step 1) with a halide compound to prepare a compound of Formula 1-2, 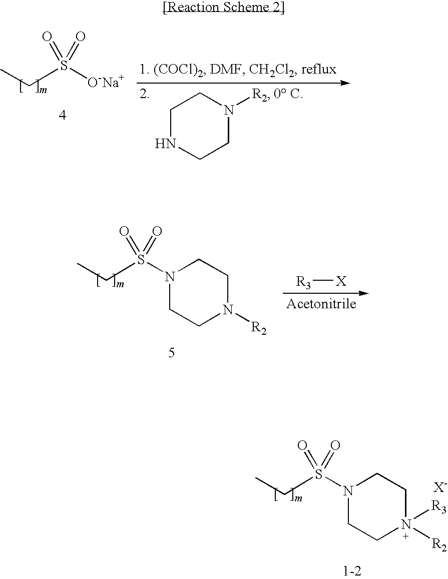
for Reaction Scheme 2,
R₂ is straight or branched chain C₁˜C₆ alkyl,
R₃ is straight or branched chain C₁˜C₆ alkyl; C₂˜C₃₀ alkenyl; allyl; or benzyl substituted or unsubstituted with one group selected from the group consisting of straight or branched chain C₁˜C₆ alkyl, C₁˜C₆ alkoxy, OCF₃, nitro, and halogen atom,
X is a halogen atom, and
m is an integer of 1 to 30.

5. A process for the preparation of the compounds of claim 1 represented by Reaction Scheme 3, the process comprising the steps of:
1) reacting an organic acid compound of Formula 2 with an alkyldiazepan derivative in an organic solvent to prepare a compound of Formula 6; and
2) reacting the compound of Formula 6 prepared in the step 1) with a halide compound to prepare a compound of Formula 1-3. 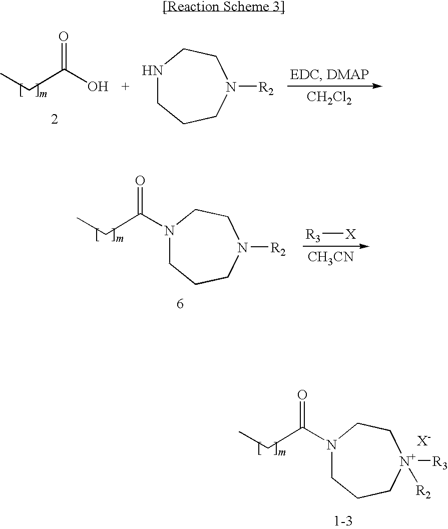
for Reaction Scheme 3,
R₂ is straight or branched chain C₁˜C₆ alkyl,
R₃ is straight or branched chain C₁˜C₆ alkyl; C₂˜C₃₀ alkenyl; allyl; or benzyl substituted or unsubstituted with one group selected from the group consisting of straight or branched chain C₁˜C₆ alkyl, C₁˜C₆ alkoxy, OCF₃, nitro, and halogen atom,
X is a halogen atom, and
m is an integer of 1 to 30.

6. A pharmaceutical composition for treatment of cancer comprising the heterocyclic compounds containing nitrogen atoms or the pharmaceutically acceptable salts thereof of claim 1 as an active ingredient.

7. The pharmaceutical composition for treatment of cancer according to claim 6, wherein the cancer is one selected from the group consisting of lung cancer, non-small cell lung cancer, colon cancer, bone cancer, pancreatic cancer, skin cancer, head or neck cancer, cutaneous or ocular melanoma, uterine cancer, ovarian cancer, rectal cancer, gastric cancer, anal cancer, breast cancer, fallopian tube carcinoma, endometrial carcinoma, cervical carcinoma, vaginal carcinoma, vulvar carcinoma, Hodgkin's disease, esophageal cancer, small intestine cancer, endocrine gland cancer, thyroid cancer, parathyroid cancer, adrenal cancer, soft-tissue sarcoma, uterine cancer, penis cancer, prostate cancer, chronic or acute leukemia, lymphocyte lymphoma, bladder cancer, kidney or ureter cancer, renal cell carcinoma, renal pelvic carcinoma, central nervous system tumor, primary central nervous system lymphoma, spinal tumor, brain stem glioma, and pituitary adenoma.

8. The pharmaceutical composition for treatment of cancer according to claim 7, wherein the cancer is prostate cancer, breast cancer, kidney cancer, or gastric cancer.
